(19) United States
(12) Patent Application Publication
(10) Pub. No.: US 2020/0121812 A1
(43) Pub. Date: Apr. 23, 2020

(54) Combination of Deuterated Levodopa With Carbidopa and Opicapone For The Treatment of Parkinson's Disease

(71) Applicant: Teva Pharmaceuticals International GmbH, Jona (CH)

(72) Inventors: Hermann Russ, Altendorf (CH); Silvia A. Mandel, Arie Sharon (IL); Aric Orbach, Rehovot (IL)

(21) Appl. No.: 16/555,915

(22) Filed: Aug. 29, 2019

Related U.S. Application Data
(63) Continuation of application No. 15/401,270, filed on Jan. 9, 2017, now abandoned.
Application No. 15/401,270 is a continuation of application No. 15/288,730, filed on Oct. 7, 2016, now abandoned.
(60) Provisional application No. 62/284,800, filed on Oct. 9, 2015.

(51) Int. Cl. A61K 51/04; A61K 31/4245; A61K 31/197; A61K 9/20; A61K 9/28; A61K 31/165; A61K 31/4439; A61K 31/198

(52) U.S. Cl. CPC A61K 51/0406; A61K 31/4245; A61K 31/197; A61K 9/2059; A61K 9/2813; A61K 9/2853; A61K 31/198; A61K 31/4439; A61K 9/2018; A61K 9/2866; A61K 9/2054; A61K 9/2027; A61K 31/165

(57) ABSTRACT

The present invention relates to new combinations of treatments for abnormal dopamine deficiency disorders, and related conditions, comprising deuterated catecholamine derivatives and catechol-O-methyltransferase (COMT) inhibitors.

### CROSS REFERENCE TO RELATED APPLICATIONS

This application is a continuation of U.S. patent application Ser. No. 15/401,270, filed Jan. 9, 2017, which is a continuation of U.S. application Ser. No. 15/288,730, filed Oct. 7, 2016, which claims the benefit of U.S. Provisional Patent Application No. 62/284,800, filed Oct. 9, 2015, all of which are incorporated by reference herein.

### FIELD OF THE DISCLOSURE

Disclosed herein are combinations of treatments for abnormal dopamine deficiency disorders, and related conditions, comprising deuterated catecholamine derivatives and catechol-O-methyltransferase (COMT) inhibitors.

### BACKGROUND

Parkinson's disease (PD) is a degenerative disorder of the central nervous system mainly affecting the motor system. The motor symptoms of Parkinson's disease result from the degeneration of dopamine-generating cells in the substantia nigra region of the central nervous system. Early in the course of the disease, the most obvious symptoms are movement-related; these include shaking, rigidity, bradykinesia (slowness of movement), resting tremor, postural reflex impairment, and difficulty with walking and gait. Additional, and often later-manifesting, symptoms include autonomic disturbances, sleep disturbances, and cognitive dysfunctions, depression, anxiety,

Levodopa (L-DOPA) remains the primary treatment for Parkinson's disease.

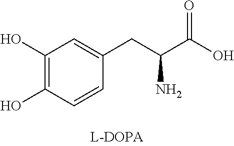 [SMILES: N[C@@H](Cc1ccc(O)c(O)c1)C(=O)O]

Levodopa is a precursor to dopamine, and is administered to Parkinson's patients to provide a replacement source for dopamine in the central nervous system (CNS). Improvement of the impaired dopaminergic neurotransmission by administration of levodopa is the backbone of the current pharmacotherapy. Patients with advanced Parkinson's disease require higher doses of dopaminergics but this therapy is limited by motor complications, like fluctuations and involuntarily movements (described as levodopa induced dyskinesia, LIDs). Fluctuations might be due to the shorter striatal persistence (half life) of dopamine especially in advanced Parkinson's disease patients.

The therapeutic effect of levodopa depends on its biotransformation to dopamine in the brain. However, levodopa undergoes rapid and extensive metabolism by peripheral aromatic L-amino acid decarboxylase (AADC) and catechol-O-methyltransferase (COMT) and only 1% of an oral dose of levodopa actually reaches the brain. Therefore, levodopa is usually co-administered with an AADC inhibitor (carbidopa or benserazide) which increases levodopa bioavailability. Even so, approximately 90% of a levodopa dose is converted by COMT to 3-O-methyl-levodopa (3-OMD), which has a long half-life compared to L-DOPA and competes with levodopa for transport across the blood-brain barrier (BBB).

Thus, an additional strategy to further inhibit peripheral levodopa metabolism and increase the delivery of levodopa to the brain is the administration of a COMT inhibitor. COMT inhibition as adjunct to levodopa/aromatic AADC inhibitor (AADCi) therapy provides pharmacodynamic benefits to Parkinson's patients. Commonly, within only a few years of starting levodopa therapy with the usual administration regime, levodopa-induced clinical improvement declines at the end of each dose cycle, giving rise to the so-called “wearing-off” pattern of motor fluctuations. A close relationship between the accumulation of 3-OMD and the wearing-off phenomenon and has been described (Tohgi, H., et al., Neurosci. Letters, 132:19-22, 1992).

Two COMT inhibitors, tolcapone and entacapone, are currently approved in the United States, and both have clinical limitations. Tolcapone crosses the BBB and potently inhibits both central and peripheral COMT. Shortly after its launch, tolcapone was withdrawn from the market after several cases of hepatotoxicity were reported including three deaths from fatal fulminant hepatitis. As a result, the use of tolcapone now requires liver function monitoring and thus is limited to fluctuating patients poorly controlled with other therapies. Entacapone is a peripherally-acting compound unable to cross the BBB, and is a significantly less potent COMT inhibitor than tolcapone and has a much shorter in-vivo half-life. Accordingly, entacapone has a very limited duration of effect and must be administered in very high doses with every dose of levodopa, making patient compliance problematic.

Opicapone (also known as 2,5-dichloro-3-[5-(3,4-dihydroxy-5-nitrophenyl]-1,2,4-oxadiazol-3-yl)-4,6-dimethylpyridine 1-oxide, 5-[3-(2,5-dichloro-4,6-dimethyl-1-oxy-pyridin-3-yl)[1,2,4]oxadiazol-5-yl]-3-nitrobenzene-1,2-diol, or BIA 9-1067)

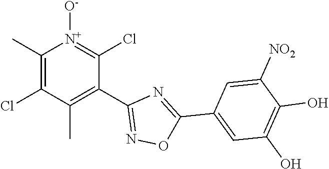 [SMILES: Cc1c(Cl)c(C)[n+]([O-])c(Cl)c1-c1noc(-c2cc(O)c(O)c([N+](=O)[O-])c2)n1]

is a third generation COMT inhibitor currently in phase III clinical trials for use as adjunctive therapy in levodopa-treated PD patients. Opicapone has a high binding affinity and a corresponding slow complex dissociation rate constant and long duration of action in vivo. In Parkinson's patients, opicapone has been shown to increase levodopa exposure in a dose-dependent manner and improve various motor outcomes.

Deuterated analogues of the catecholamine L-DOPA, discussed further below, have been prepared and have been found to have improved properties compared to L-DOPA. α,β, β-D3-L-DOPA (L-2-Amino-2,3,3-trideutero-3-(3,4-dihydroxyphenyl) propionic acid) and α,β-D2-L-DOPA (S/S-2-amino-2,3-dideutero-3-(3,4-dihydroxyphenyl) propionic acid) are two examples of such compounds:

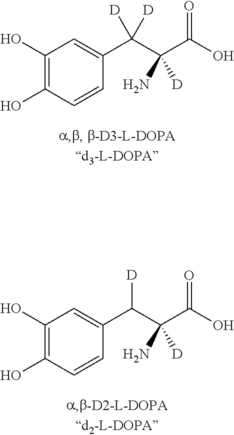 [SMILES: [2H]C([2H])(c1ccc(O)c(O)c1)[C@]([2H])(N)C(=O)O ; [2H]C(c1ccc(O)c(O)c1)[C@]([2H])(N)C(=O)O]

α,β-D2-L-DOPA comprises two enantiomers, and the stereononspecific notation above is intended to refer to either or both:

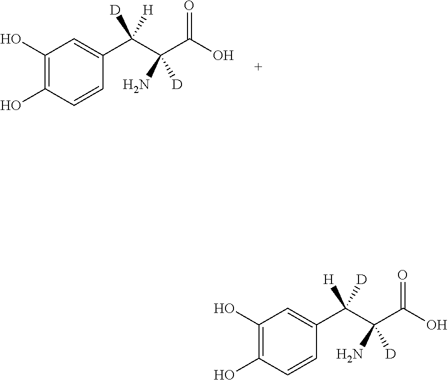 [SMILES: [2H][C@H](c1ccc(O)c(O)c1)[C@]([2H])(N)C(=O)O ; [2H][C@@H](c1ccc(O)c(O)c1)[C@]([2H])(N)C(=O)O]

For example, α,β, β-D3-L-DOPA exhibited higher longer-lasting striatal dopamine levels than L-DOPA. Correspondingly to the increased availability of dopamine in the striatum, α,β,β-D3-L-DOPA showed improved motor activity compared to L-DOPA in several Parkinson models (Malmlof et al., Exp Neurol, 2008, 538-542; Malmlof et al., Exp Neurol, 2010, 225: 408-415). The equi-effective dose of α,β,β-D3-L-DOPA compared to L-DOPA was about 60%. The observed longer striatal persistence of dopamine allowed the assumption that fluctuations might be reduced as well. Similarly, both α,β,β-D3-L-DOPA and α,β-D2-L-DOPA were shown to increase and prolong the output of striatal dopamine significantly more than L-DOPA (see, e.g., WO2004/056724 and WO2007/093450).

The highest striatal dopamine concentrations were found after administration of α,β-D2-L-DOPA. Those dopamine levels were even higher than those after the administration of the triple-deuterated α,β,β-D3-L-DOPA which included the same deuterated positions as the double deuterated L-DOPA. At the equi-effective dose (same striatal dopamine levels and same motor effect as L-DOPA), α,β,β-D3-L-DOPA caused significant less dyskinesia than L-DOPA (Malmlof et al., Exp Neural, 2010, 225: 408-415).

Composition 1 comprises α,β,β-D3-L-DOPA and α,β-D2-L-DOPA in a ratio of about 90% to about 10%:

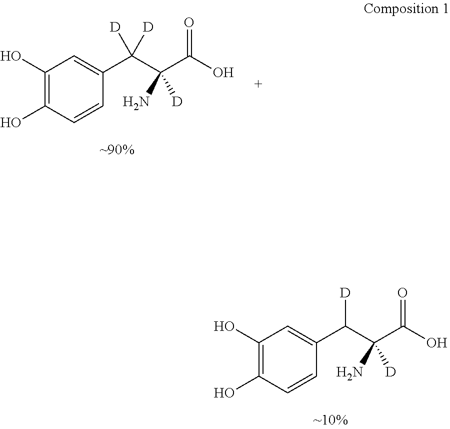 [SMILES: [2H]C([2H])(c1ccc(O)c(O)c1)[C@]([2H])(N)C(=O)O ; [2H]C(c1ccc(O)c(O)c1)[C@]([2H])(N)C(=O)O]

Composition 1 may be prepared, as will be further discussed below, by admixture of α,β,β-D3-L-DOPA and α,β-D2-L-DOPA in the stated proportions, or by addition of specifically enriched starting material to a certain step during the preparation process of the compounds. Accordingly, another way to refer to Composition 1 is

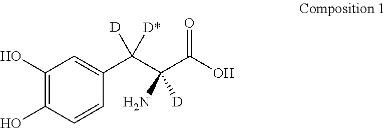

or α,β,β*-D3-L-DOPA (L-2-amino-2,3,3*-trideutero-3-(3,4-dihydroxyphenyl) propionic acid) wherein the position occupied by D*/β* has about 90% enrichment, whereas other positions occupied by deuterium have enrichment of over about 98%. D*/β* may be in the (R) or the (S) configuration. Composition 1 may also be referred to as SD-1077.

Composition 1 has been shown to yield an equivalent motor effect to levodopa at 35% of the levodopa dose, and with only 50% of the observed dyskinesia side effects, in a rat model of Parkinson's disease. See, e.g., WO2014/122184A1.

Despite the developments above in levodopa therapy, a need still exists for improved therapy for Parkinson's disease and other disorders of dopamine deficiency.

### SUMMARY

The present disclosure is directed to methods of treating of a dopamine deficiency disorder in a subject in need thereof, comprising the administration, concurrently or in any order, of opicapone and a deuterated levodopa derivative.

The disclosure is also directed to methods of treatment of Parkinson's disease in a patient in need thereof, comprising the administration, in any order, of opicapone, carbidopa or benserazide, and Composition 1

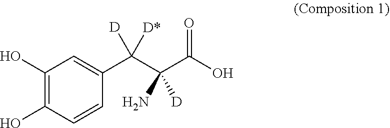

wherein each position designated D has deuterium enrichment of about 97% or more; and each position designated D* has deuterium enrichment of about 90%.

Also described are pharmaceutical compositions comprising a deuterated levodopa derivative and opicapone, together with a pharmaceutically acceptable carrier.

The disclosure is also directed to packages comprising a first pharmaceutical composition comprising an amount of a deuterated levodopa derivative and a pharmaceutically acceptable carrier; a second pharmaceutical composition comprising an amount of opicapone and a pharmaceutically acceptable carrier; and instructions for use of the first and second pharmaceutical compositions together to treat a subject afflicted with a dopamine deficiency disorder. Further disclosed are packages which include a pharmaceutical composition comprising an amount of a deuterated levodopa derivative, an amount of opicapone and a pharmaceutically acceptable carrier; and instructions for use of the pharmaceutical composition to treat a subject afflicted with a dopamine deficiency disorder.

### BRIEF DESCRIPTION OF THE DRAWINGS

FIG. 1 shows the results of a microdialysis experiment in rats. Administration of d₃-L-DOPA (50 mg/kg ip) significantly increased bioavailability of d₃-dopamine in the striatum and prolonged half life compared to conventional L-DOPA (50 mg/kg ip).

FIG. 2 shows plasma levels of levodopa and 3-OMD in wistar rats pretreated orally with opicapone or entacapone and challenged with levodopa/carbidopa.

FIG. 3 shows the results of a Rodent Model of Parkinsonian Motor Performance (6-OHDA model). Administration of Composition 1 with Opicapone demonstrated a trend for increased rotational behavior as compared with administration of conventional L-DOPA with Opicapone.

FIG. 4 shows the effects of SD-1077/Carbidopa (50/25 mg/kg p.o.) administered to rats pre-treated 2 hours before with saline, entacapone (30 mg/kg p.o.), or opicapone (30 mg/kg p.o.) on striatal dialysate levels of SD-1077.

FIG. 5 shows the effects of SD-1077/Carbidopa (50/25 mg/kg p.o.) administered to rats pre-treated 2 hours before with saline, entacapone (30 mg/kg p.o.), or opicapone (30 mg/kg p.o.) on striatal dialysate levels of deuterated-3-OMD.

FIG. 6 shows the effects of SD-1077/Carbidopa (50/25 mg/kg p.o.) administered to rats pre-treated 2 hours before with saline, entacapone (30 mg/kg p.o.), or opicapone (30 mg/kg p.o.) on striatal dialysate levels of deuterated DA.

FIG. 7 shows the effects of SD-1077/Carbidopa (50/25 mg/kg p.o.) administered to rats pre-treated 2 hours before with saline, entacapone (30 mg/kg p.o.), or opicapone (30 mg/kg p.o.) on striatal dialysate levels of deuterated DOPAC.

FIG. 8 shows the effects of SD-1077/Carbidopa (50/25 mg/kg p.o.) administered to rats pre-treated 2 hours before with saline, entacapone (30 mg/kg p.o.), or opicapone (30 mg/kg p.o.) on striatal dialysate levels of deuterated 3-MT.

### DETAILED DESCRIPTION

The major limitation of levodopa therapy is its short half life of about 1.5 hours. As a consequence, levodopa has to be taken orally several times per day (up to 7 times per day and more). That leads to a “pulsatile” stimulation of central dopamine receptors through fluctuating dopamine concentrations in the brain. This non-physiological situation is seen as a major cause for the development of so-called “motor complications” and/or dyskinesias during long-term treatment. For an optimal levodopa therapy for PD patients the drug should be applied in a way that provides constant levels of dopamine in the brain. That is currently only possible with a constant intraduodenal infusion of levodopa, which is a burdensome procedure for PD patients (Duopa in US or Duodopa in EU, AbbVie Pharma).

Disclosed herein is a new method to provide more constant levels of dopamine in the brain after intake of an oral fixed-dose combination of deuterated levodopa (in certain embodiments, with either carbidopa or benserazide) and opicapone. Opicapone is a so-called “third generation” COMT inhibitor developed by Bial Pharmaceuticals of Portugal. Opicapone has delivered positive Phase 3 results and a NDA is currently under review by the EMA. Compared to the only currently available COMT inhibitor, entacapone, opicapone shows a significantly longer duration of COMT inhibition (>8 hrs) and also acts in the brain, whereas entacapone does not.

One combination product for treatment of PD currently on the market is STALEVO® (Orion), a combination of levodopa, carbidopa and entacapone. This product has to be administered several times per day (more often in more advanced disease stages) since the half life of both major active ingredients, levodopa and entacapone, are short. As a consequence, STALEVO® reduces the OFF time significantly, but as a downside, also increases the rate of dyskinesia. Thus, combination of opicapone with deulevodopa has the potential to realize beyond known combination treatments.

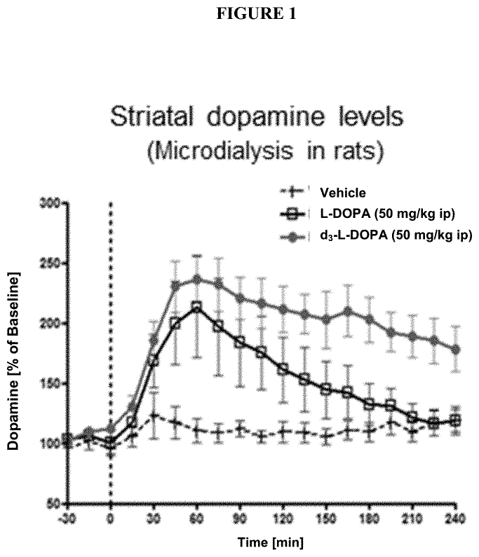

The combination product according to this invention containing as major active ingredients deulevodopa and opicapone has the following pharmacological characteristics and advantages:
Deulevodopa, after metabolism into deu-dopamine, shows a longer half life in the brain (more than doubled, see FIG. 1 with microdialysis data below)
Opicapone in addition to deu-levodopa has a two-fold effect:
   i. it increases the bioavailability of deulevodopa in plasma (entacapone-like) and
   ii. it reduces the enzymatic break down of deu-dopamine in the brain significantly (explanation below)

Both are synergistic effects leading to a reduced fluctuation of the central (striatal) dopamine levels. Through these “smoothened” dopamine levels, the pulsatile stimulation of central dopamine receptors is diminished. The therapeutic advantages of more constant central dopamine levels are less motor fluctuations and less dyskinesias.

Accordingly, provided herein is a method of treatment of a dopamine deficiency disorder in a subject in need thereof, comprising the administration, concurrently or in any order, of opicapone and a deuterated levodopa derivative.

Also provided is a method of improving motor ON time without dyskinesia in a patient with Parkinson's disease, comprising the administration, concurrently or in any order, of opicapone and a deuterated levodopa derivative.

Also provided is a method of reducing dyskinesia in a subject with a dopamine deficiency disorder, comprising the administration, concurrently or in any order, of opicapone and a deuterated levodopa derivative.

Also provided is a method of reducing motor OFF time in a subject with a dopamine deficiency disorder, comprising the administration, concurrently or in any order, of opicapone and a deuterated levodopa derivative.

Also provided is a method of reducing striatal dopamine level fluctuations in a subject with a dopamine deficiency disorder, comprising the administration, concurrently or in any order, of opicapone and a deuterated levodopa derivative.

Also provided is a method of reducing dyskinesia in a subject with a dopamine deficiency disorder after long-term treatment, comprising the administration, concurrently or in any order, of opicapone and a deuterated levodopa derivative.

Also provided is a method of reducing the rate of progression of dyskinesia. These methods comprise periodically administering to an early stage Parkinson's disease patient, an amount of opicapone and an amount of a deuterated levodopa derivative (concurrently or in any order) sufficient to reduce the rate of dyskinesia progression. Preferred methods comprise periodically administering to an early stage Parkinson's disease patient an amount of Composition 1 concurrently or in any order of opicapone, effective to reduce the rate of progression of dyskinesia of the early stage Parkinson's disease patient.

According to the disclosure, whether a patient has “early stage Parkinson's disease” can be determined by reference to the Hoehn and Yahr Scale, which is understood by those of ordinary skill in the art. Early stage Parkinson's disease according to the disclosure includes Stages 1, 2, and 3 of the Hoehn and Yahr Scale. In preferred aspects, early stage Parkinson's disease includes Stages 1 and 2 of the Hoehn and Yahr Scale.

Also provided is a method for delaying the need for symptomatic anti dyskinesia therapy in a Parkinson's disease patient. These methods comprise periodically administering to an early stage Parkinson's disease patient, an amount of opicapone and an amount of a deuterated levodopa derivative (concurrently or in any order) sufficient to delay the need for symptomatic anti dyskinesia therapy in a Parkinson's disease patient. Preferred methods comprise periodically administering to an early stage Parkinson's disease patient an amount of Composition 1 concurrently or in any order of opicapone, effective to delay the need for symptomatic anti-dyskinesia therapy

In certain embodiments, the deuterated levodopa derivative has Formula I:

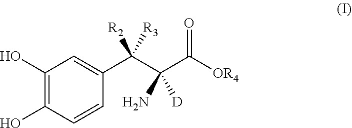

or a stereoisomer, salt, solvate, or prodrug thereof, wherein:
R₂ and R₃ are independently selected from hydrogen and deuterium, and at least one of R₂ and R₃ has a deuterium enrichment in the range from 0.02% to 100% deuterium; and wherein the deuterium enrichment of R₂ and R₃ is different from each other and that the difference between the deuterium enrichment of R₂ and R₃ is at least 5 percentage points; and
R₄ is hydrogen, deuterium, C₁ to C₆-alkyl or C₅ to C₆-cycloalkyl, deuterated C₁ to C₆-alkyl or C₅ to C₆-cycloalkyl, or a group that is easily hydrolytically or enzymatically cleavable under physiological conditions.

In certain embodiments:

R₂ is deuterium;

R₃ is selected from hydrogen and deuterium; and

R₄ is hydrogen.

In other embodiments,

R₂ is deuterium;

R₃ is selected from hydrogen and deuterium; and

R₄ is hydrogen, C₁ to C₆-alkyl, or C₅ to C₆-cycloalkyl.

In certain embodiments, the difference between the deuterium enrichment of R₂ and R₃ is about 7 to about 10 percentage points.

In certain embodiments, each position occupied by deuterium independently has deuterium enrichment of no less than about 80%.

In certain embodiments, each position occupied by deuterium independently has deuterium enrichment of no less than about 90%.

In certain embodiments, the deuterated levodopa derivative is Composition 1

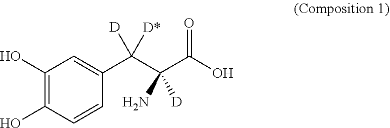

or a stereoisomer, salt, solvate, or prodrug thereof, wherein:

the position occupied by D*/β* has about 90% enrichment; and

positions occupied by D have enrichment of over 97%.

Certain embodiments of the disclosure include Composition 1, or a stereoisomer, salt, or solvate thereof.

In certain embodiments, positions occupied by D have enrichment of about 98%.

In certain embodiments, the opicapone is administered about one hour prior to administration of the deuterated levodopa derivative.

In certain embodiments, the opicapone and the deuterated levodopa derivative are administered orally.

In certain embodiments, the opicapone and the deuterated levodopa derivative are administered as one or more tablets or capsules.

In certain embodiments, the opicapone is administered without food.

In certain embodiments, the method additionally comprises the administration, concurrently or in any order, of an aromatic L-amino acid decarboxylase (“AADC”) inhibitor.

In certain embodiments, the AADC inhibitor is chosen from carbidopa and benserazide.

In certain embodiments, the AADC inhibitor is carbidopa.

In certain embodiments, the dopamine deficiency disorder is chosen from Parkinson's disease, levodopa-responsive dystonia, restless legs syndrome, neuroleptic malignant syndrome, multiple system atrophy, amyotrophic lateral sclerosis (ALS), and progressive supranuclear palsy (Steel-Richardson-Olszewski), drug-induced Parkinsonism, corticobasal degeneration, vascular Parkinsonism, Parkinsonism due to intoxication, and dementia with Lewy bodies.

In certain embodiments, the dopamine deficiency disorder is Parkinson's disease.

Also provided is a method of treatment of Parkinson's disease in a patient in need thereof, comprising the administration, in any order, of opicapone, carbidopa or benserazide, and Composition 1

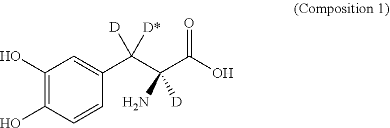

wherein

each position designated D has deuterium enrichment of about 97% or more; and

each position designated D* has deuterium enrichment of about 90%.

In certain embodiments, each position designated D has deuterium enrichment of about 98% or more

In certain embodiments, the opicapone is administered about one hour prior to administration of the Composition 1 and carbidopa or benserazide.

In certain embodiments, the opicapone, the Composition 1, and the carbidopa or benserazide are administered orally.

In certain embodiments, the opicapone, the Composition 1, and the carbidopa or benserazide are administered as one or more tablets or capsules.

In certain embodiments, the opicapone is administered without food.

In certain embodiments, the amount of Composition 1 is about 75 mg to about 6 g per day.

In certain embodiments, the amount of Composition 1 is about 25 to about 200 mg per dosage unit.

In certain embodiments, the amount of opicapone is about 5 to about 200 mg per day.

In certain embodiments, the amount of opicapone is about 5 to about 50 mg per dosage unit.

In certain embodiments, the amount of carbidopa or benserazide is about 30 to about 200 mg per day.

In certain embodiments, the amount of carbidopa or benserazide is about 10 to about 50 mg per dosage unit.

Also provided are embodiments wherein any embodiment disclosed above in the foregoing paragraphs may be combined with any one or more of these embodiments to form a new compound or class of compounds, or pharmaceutical composition comprising it, or method of use employing it, provided the combination is not mutually exclusive. For example, a combination embodiment wherein R₂ is deuterium and the disorder in need of treatment is Parkinson's disease is valid because the recited limitations are not mutually exclusive.

Also provided herein is a pharmaceutical composition comprising a deuterated levodopa derivative and opicapone, together with a pharmaceutically acceptable carrier.

In certain embodiments, the deuterated levodopa derivative has Formula I:

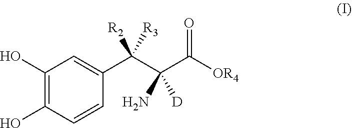

or a stereoisomer, salt, solvate, or prodrug thereof, wherein:
R₂ and R₃ are independently selected from hydrogen and deuterium, and at least one of R₂ and R₃ has a deuterium enrichment in the range from 0.02% to 100% deuterium; and wherein the deuterium enrichment of R₂ and R₃ is different from each other and that the difference between the deuterium enrichment of R₂ and R₃ is at least 5 percentage points; and
R₄ is hydrogen, deuterium, C₁ to C₆-alkyl or C₅ to C₆-cycloalkyl, deuterated C₁ to C₆-alkyl or C₅ to C₆-cycloalkyl, or a group that is easily hydrolytically or enzymatically cleavable under physiological conditions.

In certain embodiments:

R₂ is deuterium;

R₃ is selected from hydrogen and deuterium; and

R₄ is hydrogen.

In certain embodiments:

R₂ is deuterium;

R₃ is selected from hydrogen and deuterium; and
R₄ is hydrogen, C₁ to C₆-alkyl, or C₅ to C₆-cycloalkyl.

In certain embodiments, the difference between the deuterium enrichment of R2 and R3 is about 7 to about 10 percentage points.

In certain embodiments, each position occupied by deuterium independently has deuterium enrichment of no less than about 80%.

In certain embodiments, each position occupied by deuterium independently has deuterium enrichment of no less than about 90%.

In certain embodiments, the deuterated levodopa derivative is Composition 1

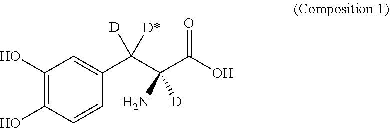

or a stereoisomer, salt, solvate, or prodrug thereof, wherein:
the position occupied by D*/β* has about 90% enrichment; and
positions occupied by D have enrichment of over 97%.

Certain embodiments of the disclosure include Composition 1, or a stereoisomer, salt, or solvate thereof.

In certain embodiments, positions occupied by D have enrichment of about 98%.

In certain embodiments, the composition comprises an immediate-release portion and a delayed-release portion, and the opicapone is in the immediate release portion, and the deuterated levodopa derivative is in the delayed-release portion, such that the deuterated levodopa derivative is absorbed about one hour after the opicapone.

In certain embodiments, the pharmaceutical composition additionally comprises an AADC inhibitor.

In certain embodiments, the AADC inhibitor is chosen from benserazide and carbidopa.

In certain embodiments, the AADC inhibitor is carbidopa.

In certain embodiments, the pharmaceutical composition is formulated as a tablet or capsule.

In certain embodiments, the tablet or capsule comprises an immediate-release portion and a delayed-release portion, and the opicapone is in the immediate release portion, and the deuterated levodopa derivative and the carbidopa or benserazide are in the delayed-release portion, such that the deuterated levodopa derivative is absorbed about one hour after the opicapone.

In certain embodiments, the amount of Composition 1 in the tablet or capsule is about 25 to about 200 mg, the amount of opicapone is about 5 to about 50 mg, and the amount of carbidopa or benserazide is about 10 to about 50 mg.

Also provided is a pharmaceutical composition as disclosed herein for use in the manufacture of a medicament for the prevention or treatment of a dopamine deficiency disorder.

In certain embodiments, the dopamine deficiency disorder is chosen from Parkinson's disease, levodopa-responsive dystonia, restless legs syndrome, neuroleptic malignant syndrome, multiple system atrophy, amyotrophic lateral sclerosis (ALS), and progressive supranuclear palsy (Steel-Richardson-Olszewski), drug-induced Parkinsonism, corticobasal degeneration, vascular Parkinsonism, Parkinsonism due to intoxication and dementia with Lewy bodies.

In certain embodiments, the dopamine deficiency disorder is Parkinson's disease.

Also provided are embodiments wherein any embodiment disclosed above in the foregoing paragraphs may be combined with any one or more of these embodiments to form a new compound or class of compounds, or pharmaceutical composition comprising it, or method of use employing it, provided the combination is not mutually exclusive.

Also provided is a package comprising:
a) a first pharmaceutical composition comprising an amount of a deuterated levodopa derivative and a pharmaceutically acceptable carrier;
b) a second pharmaceutical composition comprising an amount of opicapone and a pharmaceutically acceptable carrier; and
c) instructions for use of the first and second pharmaceutical compositions together to treat a subject afflicted with a dopamine deficiency disorder.

In certain embodiments, the dopamine deficiency disorder is chosen from Parkinson's disease, levodopa-responsive dystonia, restless legs syndrome, neuroleptic malignant syndrome, multiple system atrophy, amyotrophic lateral sclerosis (ALS), and progressive supranuclear palsy (Steel-Richardson-Olszewski), drug-induced Parkinsonism, corticobasal degeneration, vascular Parkinsonism, Parkinsonism due to intoxication and dementia with Lewy bodies.

In certain embodiments, the dopamine deficiency disorder is Parkinson's disease.

In certain embodiments, the deuterated levodopa derivative has Formula I:

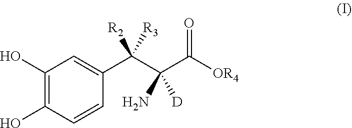

or a stereoisomer, salt, solvate, or prodrug thereof, wherein:
R₂ and R₃ are independently selected from hydrogen and deuterium, and at least one of R₂ and R₃ has a deuterium enrichment in the range from 0.02% to 100% deuterium; and wherein the deuterium enrichment of R₂ and R₃ is different from each other and that the difference between the deuterium enrichment of R₂ and R₃ is at least 5 percentage points; and
R₄ is hydrogen, deuterium, C₁ to C₆-alkyl or C₅ to C₆-cycloalkyl, deuterated C₁ to C₆-alkyl or C₅ to C₆-cycloalkyl, or a group that is easily hydrolytically or enzymatically cleavable under physiological conditions.

In some aspects:

R₂ is deuterium;

R₃ is selected from hydrogen and deuterium; and

R₄ is hydrogen.

In other aspects,

R₂ is deuterium;

R₃ is selected from hydrogen and deuterium; and

R₄ is hydrogen, C₁ to C₆-alkyl, or C₅ to C₆-cycloalkyl.

In certain embodiments, the difference between the deuterium enrichment of R² and R³ is about 7 to about 10 percentage points.

In certain embodiments, each position occupied by deuterium independently has deuterium enrichment of no less than about 80%.

In certain embodiments, each position occupied by deuterium independently has deuterium enrichment of no less than about 90%.

In certain embodiments, the deuterated levodopa derivative is Composition 1

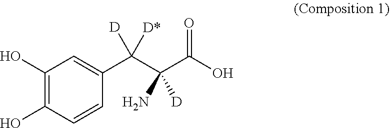

or a stereoisomer, salt, solvate, or prodrug thereof, wherein:
the position occupied by D*/β* has about 90% enrichment; and
positions occupied by D have enrichment of over 97%.

Certain embodiments of the disclosure include Composition 1, or a stereoisomer, salt, or solvate thereof.

In certain embodiments, positions occupied by D have enrichment of about 98%.

In certain embodiments, the opicapone is administered about one hour prior to administration of the deuterated levodopa derivative.

In certain embodiments, the opicapone and the deuterated levodopa derivative are administered orally.

In certain embodiments, the opicapone and the deuterated levodopa derivative are administered as one or more tablets or capsules.

In certain embodiments, the opicapone is administered without food.

In certain embodiments, the first pharmaceutical composition additionally comprises an AADC inhibitor.

In certain embodiments, the AADC inhibitor is chosen from carbidopa and benserazide.

In certain embodiments, the AADC inhibitor is carbidopa.

Also provided are embodiments wherein any embodiment disclosed above, may be combined with any one or more of these embodiments to form a new compound or class of compounds, or pharmaceutical composition comprising it, or method of use employing it, provided the combination is not mutually exclusive.

Levodopa is a catecholamine neurotransmitter. The carbon-hydrogen bonds of levodopa contain a naturally occurring distribution of hydrogen isotopes, namely ¹H or protium (about 99.9844%), ²H or deuterium (about 0.0156%), and ³H or tritium (in the range between about 0.5 and 67 tritium atoms per 10¹⁸ protium atoms). Increased levels of deuterium incorporation may produce a detectable Deuterium Kinetic Isotope Effect (DKIE) that could affect the pharmacokinetic, pharmacologic and/or toxicologic profiles of levodopa in comparison with levodopa having naturally occurring levels of deuterium.

Selective deuterium enrichment at the metabolic sites of levodopa has the potential to retard metabolism at these sites. The deuteration approach has the strong potential to slow the metabolism of levodopa and attenuate interpatient variability.

Novel pharmaceutical compositions and methods of using compounds in combination for the treatment disorders of dopamine deficiency in a patient by administering the compounds as disclosed herein.

In certain embodiments, deuterated levodopa derivative have the structures as disclosed in U.S. Pat. Nos. 8,168,820, 8,247,603, or WO2014/0122184A1.

In certain embodiments of the present invention, deuterated levodopa derivative have structural Formula I:

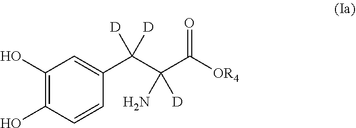

or a stereoisomer, salt, solvate, or prodrug thereof, wherein:

R₂ and R₃ are independently selected from hydrogen and deuterium, and at least one of R₂ and R₃ has a deuterium enrichment in the range from 0.02% to 100% deuterium; and wherein the deuterium enrichment of R₂ and R₃ is different from each other and that the difference between the deuterium enrichment of R₂ and R₃ is at least 5 percentage points; and

R₄ is hydrogen, deuterium, C₁ to C₆-alkyl or C₅ to C₆-cycloalkyl, deuterated C₁ to C₆-alkyl or C₅ to C₆-cycloalkyl, or a group that is easily hydrolytically or enzymatically cleavable under physiological conditions.

In certain embodiments of the present invention, deuterated levodopa derivative have structural Formula I:

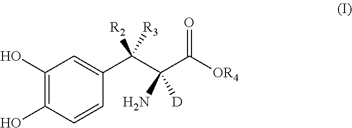

or a stereoisomer, salt, solvate, or prodrug thereof, wherein:

R₂ and R₃ are independently selected from hydrogen and deuterium, and at least one of R₂ and R₃ has a deuterium enrichment in the range from 0.02% to 100% deuterium; and wherein the deuterium enrichment of R₂ and R₃ is different from each other and that the difference between the deuterium enrichment of R₂ and R₃ is at least 5 percentage points; and

R₄ is hydrogen, deuterium, C₁ to C₆-alkyl or C₅ to C₆-cycloalkyl, deuterated C₁ to C₆-alkyl or C₅ to C₆-cycloalkyl, or a group that is easily hydrolytically or enzymatically cleavable under physiological conditions.

In certain embodiments, the difference between the deuterium enrichment of R₂ and R₃ is at least 7 percentage points. In certain embodiments, the difference between the deuterium enrichment of R₂ and R₃ is about 7 percentage points. In certain embodiments, the difference between the deuterium enrichment of R₂ and R₃ is at least 10 percentage points. In certain embodiments, the difference between the deuterium enrichment of R₂ and R₃ is about 10 percentage points. In certain embodiments, the difference between the deuterium enrichment of R₂ and R₃ is at least 15 percentage points. In certain embodiments, the difference between the deuterium enrichment of R₂ and R₃ is at least 20 percentage points.

In certain embodiments, R₄ is selected from the group comprising hydrogen, deuterium, methyl, perdeuteromethyl, ethyl, perdeuteroethyl, propyl, perdeuteropropyl, butyl, perdeuterobutyl, C₁ to C₆-alkyl, that may be branched or unbranched, or C₅ to C₆-cycloalkyl, deuterated or partly deuterated C₁ to C₆-alkyl, that may be branched or unbranched, or deuterated or partly deuterated C₅ to C₆-cycloalkyl.

In certain embodiments, R₄ is selected from the group comprising hydrogen, deuterium, methyl, perdeuteromethyl, ethyl, perdeuteroethyl, propyl, perdeuteropropyl, cyclohexyl, and perdeuterocyclohexyl.

In certain embodiments, R₄ is hydrogen.

In certain embodiments, R₄ is methyl.

In certain embodiments, R₄ is ethyl.

In certain embodiments, deuterated levodopa derivatives have structural formula IIa:

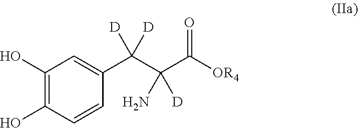

admixed with a deuterated levodopa derivative of structural Formula IIIa

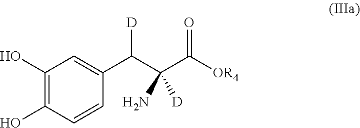

or a stereoisomer, salt, solvate, or prodrug thereof, wherein:

R₄ is hydrogen, deuterium, C₁ to C₅-alkyl or C₅ to C₆-cycloalkyl, deuterated C₁ to C₅-alkyl or C₅ to C₆-cycloalkyl, or a group that is easily hydrolytically or enzymatically cleavable under physiological conditions; and

each position designated D independently has deuterium enrichment in the range from 0.02% to 100%.

In certain embodiments, deuterated levodopa derivatives have structural formula II:

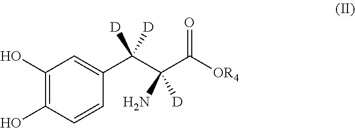

admixed with a deuterated levodopa derivative of structural Formula III and/or structural Formula IV

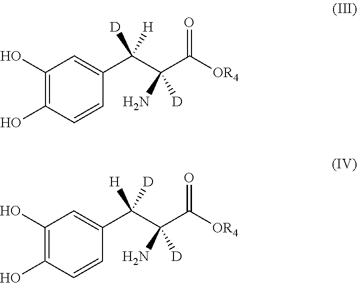

or a stereoisomer, salt, solvate, or prodrug thereof, wherein:

R₄ is hydrogen, deuterium, C₁ to C₅-alkyl or C₅ to C₆-cycloalkyl, deuterated C₁ to C₅-alkyl or C₅ to C₆-cycloalkyl, or a group that is easily hydrolytically or enzymatically cleavable under physiological conditions; and

each position designated D independently has deuterium enrichment in the range from 0.02% to 100%.

In certain embodiments, R₄ is hydrogen.

In certain embodiments, the deuterated levodopa derivative of structural Formula II is chosen from:
L-2-amino-2,3,3-trideutero-3-(3,4-dihydroxyphenyl) propionic acid,
L-2-amino-2,3,3-trideutero-3-(3,4-dihydroxyphenyl) methyl propionate,
L-2-amino-2,3,3-trideutero-3-(3,4-dihydroxyphenyl) ethyl propionate,
L-2-amino-2,3,3-trideutero-3-(3,4-dihydroxyphenyl) propyl propionate,
L-2-amino-2,3,3-trideutero-3-(3,4-dihydroxyphenyl) cyclohexyl propionate,
L-2-amino-2,3,3-trideutero-3-(3,4-dihydroxyphenyl) perdeuteromethyl propionate,
L-2-amino-2,3,3-trideutero-3-(3,4-dihydroxyphenyl) perdeuteroethyl propionate,
L-2-amino-2,3,3-trideutero-3-(3,4-dihydroxyphenyl) perdeuteropropylethyl propionate, and
L-2-amino-2,3,3-trideutero-3-(3,4-dihydroxyphenyl) perdeuterocyclohexyl propionate,
   or a stereoisomer, salt, solvate, or prodrug thereof; and wherein the deuterated levodopa derivative of structural Formula III or structural Formula IV is chosen from:
L-2-amino-2,3-dideutero-3-(3,4-dihydroxyphenyl) propionic acid,
L-2-amino-2,3-dideutero-3-(3,4-dihydroxyphenyl) methyl propionate,
L-2-amino-2,3-dideutero-3-(3,4-dihydroxyphenyl) ethyl propionate,
L-2-amino-2,3-dideutero-3-(3,4-dihydroxyphenyl) propyl propionate,
L-2-amino-2,3-dideutero-3-(3,4-dihydroxyphenyl) cyclohexyl propionate,
L-2-amino-2,3-dideutero-3-(3,4-dihydroxyphenyl) perdeuteromethyl propionate,
L-2-amino-2,3-dideutero-3-(3,4-dihydroxyphenyl) perdeuteroethyl propionate,
L-2-amino-2,3-dideutero-3-(3,4-dihydroxyphenyl) perdeuteropropylethyl propionate, and
L-2-amino-2,3-dideutero-3-(3,4-dihydroxyphenyl) perdeuterocyclohexyl propionate,
   or a stereoisomer, salt, solvate, or prodrug thereof.

In certain embodiments, the deuterated levodopa derivative of structural Formula II is chosen from:
L-2-amino-2,3,3-trideutero-3-(3,4-dihydroxyphenyl) propionic acid,
L-2-amino-2,3,3-trideutero-3-(3,4-dihydroxyphenyl) methyl propionate,
L-2-amino-2,3,3-trideutero-3-(3,4-dihydroxyphenyl) ethyl propionate,
L-2-amino-2,3,3-trideutero-3-(3,4-dihydroxyphenyl) propyl propionate,
L-2-amino-2,3,3-trideutero-3-(3,4-dihydroxyphenyl) cyclohexyl propionate, and
   or a stereoisomer, salt, solvate, or prodrug thereof; and wherein the deuterated levodopa derivative of structural Formula III or structural Formula IV is chosen from:
L-2-amino-2,3-dideutero-3-(3,4-dihydroxyphenyl) propionic acid,
L-2-amino-2,3-dideutero-3-(3,4-dihydroxyphenyl) methyl propionate,
L-2-amino-2,3-dideutero-3-(3,4-dihydroxyphenyl) ethyl propionate,
L-2-amino-2,3-dideutero-3-(3,4-dihydroxyphenyl) propyl propionate, and
L-2-amino-2,3-dideutero-3-(3,4-dihydroxyphenyl) cyclohexyl propionate,
   or a stereoisomer, salt, solvate, or prodrug thereof.

In certain embodiments, the percentage of the deuterated levodopa derivative of structural Formula II is in the range of 0.1% to 99.9%, in the range of 5% to 99%, in the range of 78% to 99%, or in the range of about 88% to about 98%. In certain embodiments, the percentage of the compound of structural Formula II is in the range of about 88% to about 92%. In certain embodiments, the percentage of the compound of structural Formula II is about 90%. In certain embodiments, the percentage of the compound of structural Formula II is in the range of about 95% to about 99%. In certain embodiments, the percentage of the compound of structural Formula II is in the range of about 96% to about 98%. In certain embodiments, the percentage of the compound of structural Formula II is about 97%. In certain embodiments, the percentage of the compound of structural Formula II is about 98%. In certain embodiments, the percentage of the compound of structural Formula II is in the range of about 78% to about 82%.

In certain embodiments, the percentage of the compound of structural Formula III and/or Formula IV is in the range of 0.1% to 99.9%, in the range of 5% to 99%, in the range of 78% to 99%, or in the range of about 88% to about 98%. In certain embodiments, the percentage of the compound of structural Formula III and/or Formula IV is in the range of about 88% to about 92%. In certain embodiments, the percentage of the compound of structural Formula III and/or Formula IV is about 90%. In certain embodiments, the percentage of the compound of structural Formula III and/or Formula IV is in the range of about 95% to about 99%. In certain embodiments, the percentage of the compound of structural Formula III and/or Formula IV is in the range of about 96% to about 98%. In certain embodiments, the percentage of the compound of structural Formula III and/or Formula IV is about 97%. In certain embodiments, the percentage of the compound of structural Formula III and/or Formula IV is about 98%. In certain embodiments, the percentage of the compound of structural Formula III and/or Formula IV is in the range of about 78% to about 82%.

In certain embodiments, each position designated D independently has deuterium enrichment of no less than about 10%. In certain embodiments, each position designated D independently has deuterium enrichment of no less than about 50%. In certain embodiments, each position designated D independently has deuterium enrichment of no less than about 70%. In certain embodiments, each position designated D independently has deuterium enrichment of no less than about 80%. In certain embodiments, each position designated D independently has deuterium enrichment of no less than about 90%. In certain embodiments, each position designated D independently has deuterium enrichment of no less than about 95%. In certain embodiments, each position designated D independently has deuterium enrichment of no less than about 96%. In certain embodiments, each position designated D independently has deuterium enrichment of no less than about 97%. In certain embodiments, each position designated D independently has deuterium enrichment of no less than about 98%. In certain embodiments, each position designated D independently has deuterium enrichment of no less than about 99%.

In certain embodiments, the deuterated catecholamine derivative is Composition 1:

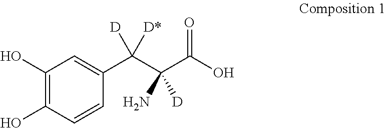

wherein the position occupied by D*/β* has about 90% deuterium enrichment, whereas other positions occupied by D have deuterium enrichment of over 97%. In certain embodiments, positions occupied by D have deuterium enrichment of about 98%.

In certain embodiments, deuterated catecholamine derivatives disclosed herein, including but not limited to Composition 1, are administered in an amount from about 25 mg to about 3 g per day. In certain embodiments, from about 100 to about 1500 mg per day. The daily amount may be administered in one dose or in divided doses of two, three, four, or more times per day. In certain embodiments, deuterated catecholamine derivatives disclosed herein, including but not limited to Composition 1, are administered in an amount of about 25, 30, 35, 40, 45, 50, 55, 60, 70, 80, 90, 100, 150, 200, 250, 300, 350, 400, 450, 500, 550, 600, 650, 700, 750, 800, 850, 900, 950, 1000, 1100, 1200, 1300, 1400, 1500, 1600, 1700, 1800 1900, 2000, 2200, or 2400 mg per day; in further embodiments, each dose may be administered either two, three, or four times per day. Each dose need not be precisely equal but will typically be similar. In certain embodiments, Composition 1 is administered in an amount of about 10 to about 500 mg per dosage unit. In certain embodiments, Composition 1 is administered in an amount from about 25 to about 200 mg per dosage unit. In certain embodiments, Composition 1 is administered in an amount of about (25, 30, 35, 40, 45, 50, 55, 60, 65, 70, 75, 100, 125, 150, 175, 200, 225, 250, 300, 350, 400, 450, or 500) mg per dosage unit, two, three, or four times per day.

In certain embodiments, opicapone is administered in a dose ranging from about 5 to about 1200 mg. In certain embodiments, opicapone is administered in an amount of about (5, 10, 15, 20, 25, 30, 35, 40, 45, 50, 100, 200, 400, 800 or 1,200) mg. In certain embodiments, opicapone is administered in an amount of about 5, about 10 mg, about 15 mg, about 20 mg, about 25 mg, about 30 mg, about 35 mg, about 40 mg, about 45 mg, or about 50 mg.

In certain embodiments, AADC inhibitors including but not limited to carbidopa and benserazide are administered in a dose ranging from about 12.5 to about 200 mg per day. In certain embodiments, carbidopa is administered in an amount of about 12.5 mg, about 25 mg, about 37.5 mg, about 50 mg, about 67.5 mg, about 75 mg, about 100 mg, about 125 mg, about 150 mg, about 175 mg, or about 200 mg per day. The daily amount may be administered in one dose or in divided doses of two, three, four, or more times per day, and will typically be formulated with or given at the same time as the deuterated catecholamine derivative.

In certain embodiments, compounds of any of Formulas I-IV can include a single enantiomer, a single diastereomer, a mixture of enantiomers (i.e., a mixture of the (+)-enantiomer and the (−)-enantiomer), a mixture of diastereomers, a mixture of about 90% or more by weight of the (−)-enantiomer and about 10% or less by weight of the (+)-enantiomer, a mixture of about 90% or more by weight of the (+)-enantiomer and about 10% or less by weight of the (−)-enantiomer, an individual diastereomer, or a mixture of diastereomers thereof.

The compounds as disclosed herein may also contain less prevalent isotopes for other elements, including, but not limited to, ¹¹C or ¹⁴C for carbon ¹⁵N for nitrogen, and ¹⁷O or ¹⁸O for oxygen.

In certain embodiments, the deuterated compounds disclosed herein maintain the beneficial aspects of the corresponding non-isotopically enriched molecules while substantially increasing the maximum tolerated dose, decreasing toxicity, increasing the half-life (T_{1/2}), lowering the maximum plasma concentration (Cₘₐₓ) of the minimum efficacious dose (MED), lowering the efficacious dose and thus decreasing the non-mechanism-related toxicity, and/or lowering the probability of drug-drug interactions.

All publications and references cited herein are expressly incorporated herein by reference in their entirety. However, with respect to any similar or identical terms found in both the incorporated publications or references and those explicitly put forth or defined in this document, then those terms definitions or meanings explicitly put forth in this document shall control in all respects.

As used herein, the terms below have the meanings indicated.

The singular forms “a,” “an,” and “the” may refer to plural articles unless specifically stated otherwise.

The term “about,” as used herein, is intended to qualify the numerical values which it modifies, denoting such a value as variable within a margin of error. When no particular margin of error, such as a standard deviation to a mean value given in a chart or table of data, is recited, the term “about” should be understood to mean that range which would encompass the recited value and the range which would be included by rounding up or down to that figure as well, taking into account significant figures.

The term “deuterium enrichment” refers to the percentage (equivalent to mol %) of incorporation of deuterium at a given position in a molecule in the place of hydrogen. For example, deuterium enrichment of 1% at a given position means that 1% of molecules in a given sample contain deuterium at the specified position. Because the naturally occurring distribution of deuterium is about 0.0156%, deuterium enrichment at any position in a compound synthesized using non-enriched starting materials is about 0.0156%. The deuterium enrichment can be determined using conventional analytical methods known to one of ordinary skill in the art, including mass spectrometry and nuclear magnetic resonance spectroscopy.

The term “is/are deuterium,” when used to describe a given position in a molecule or the symbol “D”, when used to represent a given position in a drawing of a molecular structure, means that the specified position is enriched with deuterium above the naturally occurring distribution of deuterium. In one embodiment deuterium enrichment is no less than about 1%, in another no less than about 5%, in another no less than about 10%, in another no less than about 20%, in another no less than about 50%, in another no less than about 70%, in another no less than about 80%, in another no less than about 90%, or in another no less than about 98% of deuterium at the specified position.

The term “isotopic enrichment” refers to the percentage of incorporation of a less prevalent isotope of an element at a given position in a molecule in the place of the more prevalent isotope of the element.

The term “non-isotopically enriched” refers to a molecule in which the percentages of the various isotopes are substantially the same as the naturally occurring percentages.

Asymmetric centers exist in the compounds disclosed herein. These centers are designated by the symbols “R” or “S,” depending on the configuration of substituents around the chiral carbon atom. It should be understood that the invention encompasses all stereochemical isomeric forms, including diastereomeric, enantiomeric, and epimeric forms, as well as D-isomers and L-isomers, and mixtures thereof. Individual stereoisomers of compounds can be prepared synthetically from commercially available starting materials which contain chiral centers or by preparation of mixtures of enantiomeric products followed by separation such as conversion to a mixture of diastereomers followed by separation or recrystallization, chromatographic techniques, direct separation of enantiomers on chiral chromatographic columns, or any other appropriate method known in the art. Starting compounds of particular stereochemistry are either commercially available or can be made and resolved by techniques known in the art. Additionally, the compounds disclosed herein may exist as geometric isomers. The present invention includes all cis, trans, syn, anti, entgegen (E), and zusammen (Z) isomers as well as the appropriate mixtures thereof. Additionally, compounds may exist as tautomers; all tautomeric isomers are provided by this invention. Additionally, the compounds disclosed herein can exist in unsolvated as well as solvated forms with pharmaceutically acceptable solvents such as water, ethanol, and the like. In general, the solvated forms are considered equivalent to the unsolvated forms.

The term “bond” refers to a covalent linkage between two atoms, or two moieties when the atoms joined by the bond are considered to be part of larger substructure. A bond may be single, double, or triple unless otherwise specified. A dashed line between two atoms in a drawing of a molecule indicates that an additional bond may be present or absent at that position.

The term “disorder” as used herein is intended to be generally synonymous, and is used interchangeably with, the terms “disease”, “syndrome”, and “condition” (as in medical condition), in that all reflect an abnormal condition of the human or animal body or of one of its parts that impairs normal functioning, is typically manifested by distinguishing signs and symptoms.

The terms “treat,” “treating,” and “treatment” are meant to include alleviating or abrogating a disorder or one or more of the symptoms associated with a disorder; or alleviating or eradicating the cause(s) of the disorder itself. As used herein, reference to “treatment” of a disorder is intended to include prevention. The terms “prevent,” “preventing,” and “prevention” refer to a method of delaying or precluding the onset of a disorder; and/or its attendant symptoms, barring a subject from acquiring a disorder or reducing a subject's risk of acquiring a disorder.

The term “therapeutically effective amount” refers to the amount of a compound that, when administered, is sufficient to prevent development of, or alleviate to some extent, one or more of the symptoms of the disorder being treated. The term “therapeutically effective amount” also refers to the amount of a compound that is sufficient to elicit the biological or medical response of a cell, tissue, system, animal, or human that is being sought by a researcher, veterinarian, medical doctor, or clinician.

The term “subject” refers to an animal, including, but not limited to, a primate (e.g., human, monkey, chimpanzee, gorilla, and the like, preferably human), rodents (e.g., rats, mice, gerbils, hamsters, ferrets, and the like), lagomorphs, swine (e.g., pig, miniature pig), equine, canine, feline, and the like. The terms “subject” and “patient” are used interchangeably herein in reference, for example, to a mammalian subject, such as a human patient.

The term “combination therapy” means the administration of two or more therapeutic agents to treat a therapeutic disorder described in the present disclosure. Such administration encompasses co-administration of these therapeutic agents in a substantially simultaneous manner, such as in a single capsule having a fixed ratio of active ingredients or in multiple, separate capsules for each active ingredient. In addition, such administration also encompasses use of each type of therapeutic agent in a sequential manner, in any order. In either case, the treatment regimen will provide beneficial effects of the drug combination in treating the disorders described herein.

As used herein, the term “dopamine” can include both natural dopamine and dopamine formed from a deuterated levodopa derivative, such as Composition 1. This is particularly so when striatal dopamine levels are reported and not explicitly compared to non-deuterated dopamine. Both are expected to provide therapy for dopamine deficiency disorders.

The term “dopamine deficiency disorder” refers to disorders wherein chronic deficiency of dopamine in the central nervous system is part of the pathology of the disorder and/or can be treated with levodopa or dopaminergig drugs. Such disorders may involve impairment or dopamine-producing cells in the central nervous system, and/or disrupted tyrosine or levodopa transport or disrupted tyrosine decarboxylase or DOPA-decarboxylase activity. Dopamine deficiency disorders include, without limitation, Parkinson's disease, levodopa-responsive dystonia (also known as dopamine-responsive dystonia, hereditary progressive dystonia with diurnal fluctuation, Segawa's disease, and Segawa's dystonia), restless legs syndrome, neuroleptic malignant syndrome, multiple system atrophy, amyotrophic lateral sclerosis (ALS), and progressive supranuclear palsy (Steel-Richardson-Olszewski), as well as all other forms of atypical Parkinson syndromes including drug-induced Parkinsonism, corticobasal degeneration, vascular Parkinsonism, Parkinsonism due to intoxication (e.g., from manganese, MPTP, etc.) and dementia with Lewy bodies. In certain embodiments, the dopamine deficiency disorder is Parkinson's disease. The methods and compositions disclosed herein are also useful for inhibiting prolactin secretion, for stimulating the release of growth hormone.

The term “reducing striatal dopamine level fluctuations” as used herein should be understood to be synonymous with smoothening, or reducing striatal dopamine peak-to-trough ratio in, a time-vs.-concentration curve (in pharmacokinetic terms), and reduction of pulsatile dopamine receptor stimulation (in therapeutic terms). All refer to providing a more constant level of dopamine in the brain of a subject, such that low levels of dopamine, often associated with OFF-time, and high levels of dopamine, often associated with side effects such as dyskinesia, are avoided.

The term “therapeutically acceptable” refers to those compounds (or salts, prodrugs, tautomers, zwitterionic forms, etc.) which are suitable for use in contact with the tissues of patients without excessive toxicity, irritation, allergic response, immunogenecity, are commensurate with a reasonable benefit/risk ratio, and are effective for their intended use.

The term “pharmaceutically acceptable carrier,” “pharmaceutically acceptable excipient,” “physiologically acceptable carrier,” or “physiologically acceptable excipient” refers to a pharmaceutically-acceptable material, composition, or vehicle, such as a liquid or solid filler, diluent, excipient, solvent, or encapsulating material. Each component must be “pharmaceutically acceptable” in the sense of being compatible with the other ingredients of a pharmaceutical formulation. It must also be suitable for use in contact with the tissue or organ of humans and animals without excessive toxicity, irritation, allergic response, immunogenecity, or other problems or complications, commensurate with a reasonable benefit/risk ratio.

The terms “active ingredient,” “active compound,” and “active substance” refer to a compound, which is administered, alone or in combination with one or more pharmaceutically acceptable excipients or carriers, to a subject for treating, preventing, or ameliorating one or more symptoms of a disorder.

The terms “drug,” “therapeutic agent,” and “chemotherapeutic agent” refer to a compound, or a pharmaceutical composition thereof, which is administered to a subject for treating, preventing, or ameliorating one or more symptoms of a disorder.

The term “prodrug” refers to a compound functional derivative of the compound as disclosed herein and is readily convertible into the parent compound in vivo. Prodrugs are often useful because, in some situations, they may be easier to administer than the parent compound. They may, for instance, be bioavailable by oral administration whereas the parent compound is not. The prodrug may also have enhanced solubility in pharmaceutical compositions over the parent compound. A prodrug may be converted into the parent drug by various mechanisms, including enzymatic processes and metabolic hydrolysis.

The compounds disclosed herein can exist as therapeutically acceptable salts. The term “therapeutically acceptable salt,” as used herein, represents salts or zwitterionic forms of the compounds disclosed herein which are therapeutically acceptable as defined herein. The salts can be prepared during the final isolation and purification of the compounds or separately by reacting the appropriate compound with a suitable acid or base. Therapeutically acceptable salts include acid and basic addition salts.

Suitable acids for use in the preparation of pharmaceutically acceptable salts include, but are not limited to, acetic acid, 2,2-dichloroacetic acid, acylated amino acids, adipic acid, alginic acid, ascorbic acid, L-aspartic acid, benzenesulfonic acid, benzoic acid, 4-acetamidobenzoic acid, boric acid, (+)-camphoric acid, camphorsulfonic acid, (+)-(1S)-camphor-10-sulfonic acid, capric acid, caproic acid, caprylic acid, cinnamic acid, citric acid, cyclamic acid, cyclohexanesulfamic acid, dodecyl sulfuric acid, ethane-1,2-disulfonic acid, ethanesulfonic acid, 2-hydroxy-ethanesulfonic acid, formic acid, fumaric acid, galactaric acid, gentisic acid, glucoheptonic acid, D-gluconic acid, D-glucuronic acid, L-glutamic acid, α-oxo-glutaric acid, glycolic acid, hippuric acid, hydrobromic acid, hydrochloric acid, hydroiodic acid, (+)-L-lactic acid, (±)-DL-lactic acid, lactobionic acid, lauric acid, maleic acid, (−)-L-malic acid, malonic acid, (±)-DL-mandelic acid, methanesulfonic acid, naphthalene-2-sulfonic acid, naphthalene-1,5-disulfonic acid, 1-hydroxy-2-naphthoic acid, nicotinic acid, nitric acid, oleic acid, orotic acid, oxalic acid, palmitic acid, pamoic acid, perchloric acid, phosphoric acid, L-pyroglutamic acid, saccharic acid, salicylic acid, 4-amino-salicylic acid, sebacic acid, stearic acid, succinic acid, sulfuric acid, tannic acid, (+)-L-tartaric acid, thiocyanic acid, p-toluenesulfonic acid, undecylenic acid, and valeric acid.

Suitable bases for use in the preparation of pharmaceutically acceptable salts, including, but not limited to, inorganic bases, such as magnesium hydroxide, calcium hydroxide, potassium hydroxide, zinc hydroxide, or sodium hydroxide; and organic bases, such as primary, secondary, tertiary, and quaternary, aliphatic and aromatic amines, including L-arginine, benethamine, benzathine, choline, deanol, diethanolamine, diethylamine, dimethylamine, dipropylamine, diisopropylamine, 2-(diethylamino)-ethanol, ethanolamine, ethylamine, ethylenediamine, isopropylamine, N-methyl-glucamine, hydrabamine, 1H-imidazole, L-lysine, morpholine, 4-(2-hydroxyethyl)-morpholine, methylamine, piperidine, piperazine, propylamine, pyrrolidine, 1-(2-hydroxyethyl)-pyrrolidine, pyridine, quinuclidine, quinoline, isoquinoline, secondary amines, triethanolamine, trimethylamine, triethylamine, N-methyl-D-glucamine, 2-amino-2-(hydroxymethyl)-1,3-propanediol, and tromethamine.

While it may be possible for the compounds of the subject invention to be administered as the raw chemical, it is also possible to present them as a pharmaceutical composition. Accordingly, provided herein are pharmaceutical compositions which comprise one or more of certain compounds disclosed herein, or one or more pharmaceutically acceptable salts, prodrugs, or solvates thereof, together with one or more pharmaceutically acceptable carriers thereof and optionally one or more other therapeutic ingredients. Proper formulation is dependent upon the route of administration chosen. Any of the well-known techniques, carriers, and excipients may be used as suitable and as understood in the art. The pharmaceutical compositions disclosed herein may be manufactured in any manner known in the art, e.g., by means of conventional mixing, dissolving, granulating, dragee-making, levigating, emulsifying, encapsulating, entrapping or compression processes. The pharmaceutical compositions may also be formulated as a modified release dosage form. These dosage forms can be prepared of conventional methods and techniques known to those skilled in the art.

The compositions include those suitable for oral, rectal, and topical (including dermal, buccal, sublingual and intraocular) administration although the most suitable route may depend upon for example the condition and disorder of the recipient. The compositions may conveniently be presented in unit dosage form and may be prepared by any of the methods well known in the art of pharmacy. Typically, these methods include the step of bringing into association a compound of the subject invention or a pharmaceutically salt, prodrug, or solvate thereof (“active ingredient”) with the carrier which constitutes one or more accessory ingredients. In general, the compositions are prepared by uniformly and intimately bringing into association the active ingredient with liquid carriers or finely divided solid carriers or both and then, if necessary, shaping the product into the desired formulation.

Formulations of the compounds disclosed herein suitable for oral administration may be presented as discrete units such as capsules, cachets or tablets each containing a predetermined amount of the active ingredient; as a powder or granules; as a solution or a or a non-aqueous liquid; or as an oil-in-water liquid emulsion or a water-in-oil liquid emulsion. The active ingredient may also be presented as a bolus, electuary or paste.

Pharmaceutical preparations which can be used orally include tablets, push-fit capsules made of gelatin, as well as soft, sealed capsules made of gelatin and a plasticizer, such as glycerol or sorbitol. Tablets may be made by compression or molding, optionally with one or more accessory ingredients. Compressed tablets may be prepared by compressing in a suitable machine the active ingredient in a free-flowing form such as a powder or granules, optionally mixed with binders, inert diluents, or lubricating, surface active or dispersing agents. Molded tablets may be made by molding in a suitable machine a mixture of the powdered compound moistened with an inert liquid diluent. The tablets may optionally be coated or scored and may be formulated so as to provide slow or controlled release of the active ingredient therein. All formulations for oral administration should be in dosages suitable for such administration. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

For buccal or sublingual administration, the compositions may take the form of tablets, lozenges, pastilles, or gels formulated in conventional manner. Such compositions may comprise the active ingredient in a flavored basis such as sucrose and acacia or tragacanth.

The compounds may also be formulated in rectal compositions such as suppositories or retention enemas, e.g., containing conventional suppository bases such as cocoa butter, polyethylene glycol, or other glycerides.

Certain compounds disclosed herein may be administered topically, that is by non-systemic administration. This includes the application of a compound disclosed herein externally to the epidermis or the buccal cavity and the instillation of such a compound into the ear, eye and nose, such that the compound does not significantly enter the blood stream. In contrast, systemic administration refers to oral, intravenous, intraperitoneal and intramuscular administration.

Formulations suitable for topical administration include liquid or semi-liquid preparations suitable for penetration through the skin to the site of inflammation such as gels, liniments, lotions, creams, ointments or pastes, and drops suitable for administration to the eye, ear or nose.

For administration by inhalation, compounds may be delivered from an insufflator, nebulizer pressurized packs or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Alternatively, for administration by inhalation or insufflation, the compounds of the invention may take the form of a dry powder composition, for example a powder mix of the compound and a suitable powder base such as lactose or starch. The powder composition may be presented in unit dosage form, in for example, capsules, cartridges, gelatin or blister packs from which the powder may be administered with the aid of an inhalator or insufflator.

Preferred unit dosage formulations are those containing an effective dose, as herein below recited, or an appropriate fraction thereof, of the active ingredient.

Compounds may be administered at a dose of from 0.1 to 500 mg/kg per day. Tablets or other forms of presentation provided in discrete units may conveniently contain an amount of one or more compounds which is effective at such dosage or as a multiple of the same, for instance, units containing 5 mg to 1000 mg, usually around 10 mg to 300 mg.

The amount of active ingredient that may be combined with the carrier materials to produce a single dosage form will vary depending upon the host treated and the particular mode of administration.

The compounds can be administered in various modes, e.g. orally, topically, etc. The precise amount of compound administered to a patient will be the responsibility of the attendant physician. The specific dose level for any particular patient will depend upon a variety of factors including the activity of the specific compound employed, the age, body weight, general health, sex, diets, time of administration, route of administration, rate of excretion, drug combination, the precise disorder being treated, and the severity of the disorder being treated. Also, the route of administration may vary depending on the disorder and its severity.

Compounds may be administered chronically, that is, for an extended period of time, including throughout the duration of the patient's life in order to ameliorate or otherwise control or limit the symptoms of the patient's disorder.

In the case wherein the patient's status does improve, upon the doctor's discretion the administration of the compounds may be given continuously or temporarily suspended for a certain length of time (i.e., a “drug holiday”).

Once improvement of the patient's conditions has occurred, a maintenance dose is administered if necessary. Subsequently, the dosage or the frequency of administration, or both, can be reduced, as a function of the symptoms, to a level at which the improved disorder is retained. Patients can, however, require intermittent treatment on a long-term basis upon any recurrence of symptoms.

Disclosed herein are methods of treating a dopamine deficiency disorder comprising administering to a subject having or suspected of having such a disorder, a therapeutically effective amount of a combination of a deuterated analogue of levodopa and opicapone as disclosed herein, or a pharmaceutically acceptable salt, solvate, or prodrug of either of the foregoing, or a stereoisomer thereof.

In certain embodiments, a method of treating a dopamine deficiency disorder comprises administering to the subject a therapeutically effective amount of a combination of a deuterated analogue of levodopa and opicapone as disclosed herein, or a pharmaceutically acceptable salt, solvate, or prodrug of either of the foregoing, or a stereoisomer thereof, so as to effect: (1) decreased inter-individual variation in plasma levels of the compound or a metabolite thereof; (2) increased average plasma levels of the compound or decreased average plasma levels of at least one metabolite of the compound per dosage unit; (3) at least one clinically meaningful improved disorder-control endpoint; or (4) an improved clinical effect during the treatment of the disorder, as compared to the corresponding non-isotopically enriched compound. The dopamine deficiency disorder may involve impairment or dopamine-producing cells in the central nervous system, and/or disrupted tyrosine or levodopa transport or disrupted tyrosine decarboxylase or DOPA-decarboxylase activity. Dopamine deficiency disorders include, without limitation, Parkinson's disease, levodopa-responsive dystonia, restless legs syndrome, neuroleptic malignant syndrome, multiple system atrophy, amyotrophic lateral sclerosis (ALS), and progressive supranuclear palsy (Steel-Richardson-Olszewski), as well as all other forms of atypical Parkinson syndromes including drug-induced Parkinsonism, corticobasal degeneration, vascular Parkinsonism, Parkinsonism due to intoxication (e.g., from manganese, MPTP, etc.) and dementia with Lewy bodies.

Examples of improved disorder-control and/or disorder-eradication endpoints, or improved clinical effects include, but are not limited to, change from baseline in the Unified Parkinson's Disease Rating Scale (UPDRS) or one or more subscales thereof, e.g., the motor score; more frequent and longer motor ON periods; reduced duration to ON; improved patient and clinician global impression of change; and reduced AIMS involuntary movement scores.

Besides being useful for human treatment, certain compounds and formulations disclosed herein may also be useful for veterinary treatment of companion animals, exotic animals and farm animals, including mammals, rodents, and the like. More preferred animals include horses, dogs, monkeys, and cats.

### Combination Therapy

Combination therapies are disclosed herein which are useful in the treatment of dopamine deficiency disorders. The therapeutic effectiveness of either one of the compounds described herein may be enhanced by administration of an adjuvant (i.e., by itself the adjuvant may only have minimal therapeutic benefit, but in combination with another therapeutic agent, the overall therapeutic benefit to the patient is enhanced).

Such other agents, adjuvants, or drugs, may be administered, by a route and in an amount commonly used therefor, simultaneously or sequentially with a compound as disclosed herein. When a compound as disclosed herein is used contemporaneously with one or more other drugs, a pharmaceutical composition containing such other drugs in addition to the compound disclosed herein may be utilized, but is not required.

Thus, in another aspect, certain embodiments provide methods for treating dopamine deficiency disorders in a subject in need of such treatment comprising administering to said subject an amount of a compound disclosed herein effective to reduce the symptoms and/or progression of said disorder in the subject, in combination with at least one additional agent for the treatment of said disorder. In a related aspect, certain embodiments provide therapeutic compositions comprising at least one compound disclosed herein in combination with one or more additional agents for the treatment of dopamine deficiency disorders.

### General Synthetic Methods for Preparing Compounds

Isotopic hydrogen can be introduced into a compound as disclosed herein by synthetic techniques that employ deuterated reagents, whereby incorporation rates are pre-determined; and/or by exchange techniques, wherein incorporation rates are determined by equilibrium conditions, and may be highly variable depending on the reaction conditions. Synthetic techniques, where tritium or deuterium is directly and specifically inserted by tritiated or deuterated reagents of known isotopic content, may yield high tritium or deuterium abundance, but can be limited by the chemistry required. Exchange techniques, on the other hand, may yield lower tritium or deuterium incorporation, often with the isotope being distributed over many sites on the molecule.

The compounds as disclosed herein can be prepared by methods known to one of skill in the art and routine modifications thereof, and/or following procedures similar to those described in the Example section herein and routine modifications thereof, and/or procedures found in DaSilva et al., Appl. Radiat. Isot., 1993, 44(4), 673-676; Popp et al., J. Pharm. Sci., 1978, 67(6), 871-873; Ivanov et al., Heterocycles 2001, 55(8), 1569-1572; U.S. Pat. Nos. 2,830,993; 3,045,021; WO 2007/130365; WO 2008/058261, which are hereby incorporated in their entirety, and references cited therein and routine modifications thereof. Compounds as disclosed herein can also be prepared as shown in any of the following schemes and routine modifications thereof.

The invention is further illustrated by the following examples. All IUPAC names were generated using CambridgeSoft's ChemDraw 11.0.

### Preparation of Compounds

The preparation of deuterated catecholamine derivatives can be performed in at least two principal ways. One way is to mix compounds with a certain deuterium enrichment with compounds which have only hydrogen or only a highly enriched (>98% D) deuterium substitution at a certain position. By mixing at least two compounds any required enrichment level of deuterium at any position can be obtained. The other way of preparation is to add specifically enriched starting material to a certain step during the preparation process of the compounds of the invention.

The preparation of deuterium enriched catecholamine derivatives is disclosed in WO-A 2004/056724 and WO-A 2007/093450. As disclosed therein, the preparation of selectively deuterated DOPA derivatives is disclosed that have a deuterium enrichment in the respective position within the molecule of at least 98%.

One preferred synthetic pathway is shown in Scheme 1. Deuterated catecholamine derivatives may be prepared by adding non-deuterated educts 3a and/or 4 and/or 5 to the respective deuterated compounds. The ratio of deuterated and non-deuterated compounds is adjusted in such a manner to obtain the desired ratio in the end product. This method of production has the advantage that no further mixing steps are required. This obtained product is then by definition no longer a mixture.

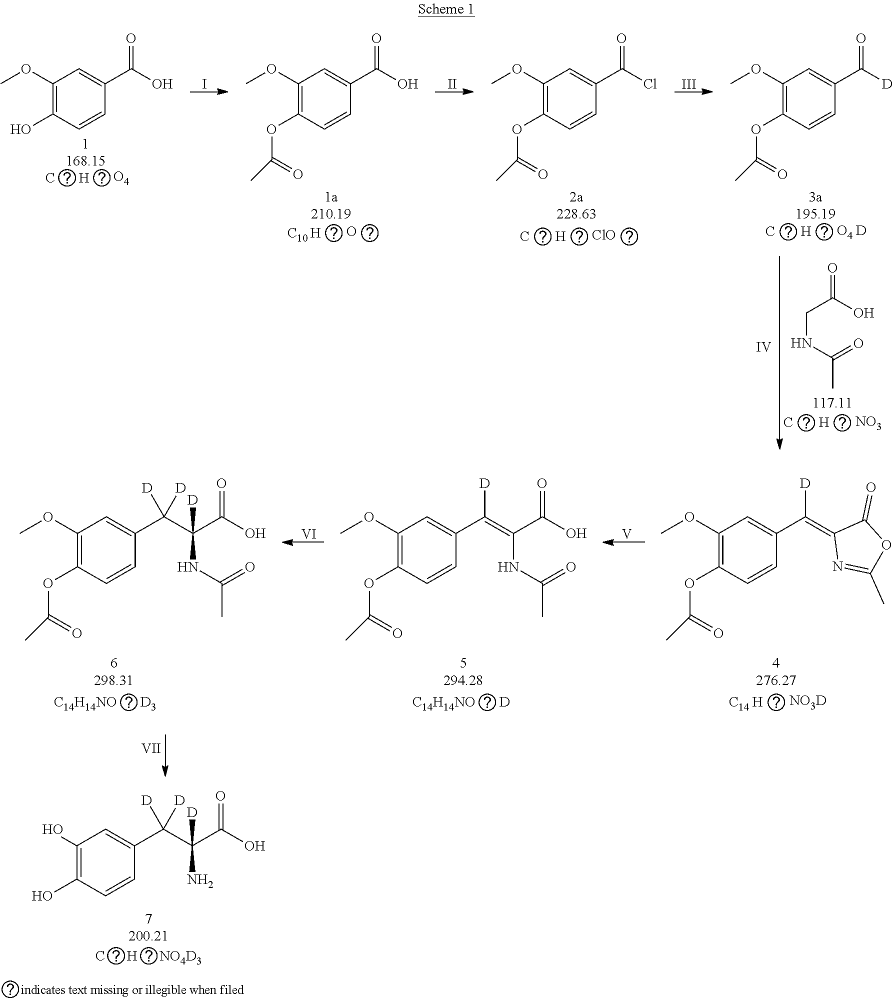

Esters of the compounds above may be made by methods known in the art, such as by using a suitable acid catalyst and alcohol, optionally with appropriate protection steps.

### Preparation per Scheme 1 of Composition 1: α,β,β*-D3-L-DOPA (L-2-amino-2,3,3*-trideutero-3-(3,4-dihydroxyphenyl) propionic acid)

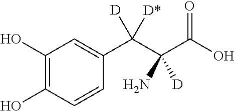

Composition 1 having a deuterium enrichment of 90% in the β* position indicated by D* is obtained by the method disclosed above.

### Preparation of Composition 1: α,β,β*-D3-L-DOPA (L-2-amino-2,3,3*-trideutero-3-(3,4-dihydroxyphenyl) propionic acid)

Alternatively, Composition 1 having a deuterium enrichment of 90% in β* position indicated by D* is obtained by mixing 10% L-2-amino-2,3(S)-dideutero-3-(3,4-dihydroxyphenyl) propionic acid with 90% L-2-amino-2,3,3-trideutero-3-(3,4-dihydroxyphenyl) propionic acid (deuterium enrichment >98% in all three positions). See, e.g., WO2014/122184A1.

Experimental Data for C₉H_{8.1}²H_{2.9}NO₄

| | | | | |
|---|---|---|---|---|
| Calculated: | H 6.95 | C 54.05 | N 7.00 | O 32.00 |
| Analyt.: | H 7.00 | C 54.02 | N 7.00 | O 31.98 |

The degree of deuteration has also been determined by NMR spectroscopy. For that purpose NMR spectra with a 500 MHz spectrometer have been recorded. As a solvent, d6-DMSO was used. The following Table 3 shows the respective position within the compound of test item D and the integral (AUC=area under curve) of the registered spectra, reflecting the content of hydrogen at the respective positions.

### NMR Results

| Position | Integral (AUC) |
|---|---|
| Ring | 3.02 |
| α | 0.02 |
| β | 0.01 |
| β* | 0.10 |

The preparation of the starting material L-2-Amino-2,3,3-trideutero-3-(3,4-dihydroxyphenyl) propionic acid is described in WO2004/056724A1, the preparation of the starting material L-2-Amino-2,3(S)-dideutero-3-(3,4-dihydroxyphenyl) propionic acid is described in WO2007/093450A1.

After mixing the compounds the mixture may be processed further in order to obtain a suitable pharmaceutical product for the medication of Parkinson's disease as given in the following examples.

The preparation of opicapone (5-[3-(2,5-Dichloro-4,6-dimethyl-1-oxy-pyridin-3-yl)-[1,2,4]oxadiazol-5-yl]-3-nitrobenzene-1,2-diol, referred to therein as “Compound A”) is disclosed in US2014/0045900A1.

Step 1.

To a stirred solution of 3,4-dibenzyloxy-5-nitrobenzoic acid (0.50 g, 1.319 mmol) in dimethylformamide (5 mL) at room temperature was added 1,1-carbonyldiimidazole (0.24 g, 1.45 mmol) in one portion. After stirring for ninety minutes, 2,5-dichloro-N′-hydroxy-4,6-dimethylnicotinamide (0.40 g, 1.45 mmol) was added in one portion. The resulting mixture was stirred at 135° C. for five hours and then at room temperature overnight. The reaction mixture was poured onto ice-2 N HCl (100 mL) and the resulting precipitate was filtered off, washed with water and dried in air. Recrystallisation from isopropanol gave a pale yellow solid (0.55 g, 72%).

Step 2.

To a stirred solution of the solid obtained above (0.50 g, 0.866 mmol) in dichloromethane (20 mL) was added urea-hydrogen peroxide addition complex (0.41 g, 4.33 mmol) in one portion. The mixture was cooled in an ice-water bath and trifluoroacetic anhydride (0.73 g, 3.46 mmol) was added dropwise. The reaction mixture was allowed to stir at room temperature overnight whereupon insoluble material was filtered off. The filtrate was washed with water and brine, dried over anhydrous magnesium sulphate, filtered and evaporated. The residue was crystallised from isopropanol to give a pale yellow solid (0.35 g, 68%).

Step 3.

To a stirred solution of the solid obtained above (0.30 g, 0.5 mmol) in dichloromethane (10 mL) at −78° C. under argon was added boron tribromide (0.38 g, 1.5 mmol) dropwise. The resulting purple suspension was allowed to stir at room temperature for one hour, then cooled again to −78° C. and carefully quenched by the addition of water. After stirring at room temperature for one hour, the precipitate was filtered off, washed with water and dried at 50° C. under vacuum to afford the desired compound as yellow crystals (0.18 g, 87%) of m.p. 237-240° C.

Additional compounds and compositions can generally be made using the methods described above.

### FORMULATION EXAMPLES

### Formulations of Deuterated Catecholamine Derivatives

The following formulations serve as examples for formulating deuterated catecholamine derivatives. See, e.g., WO2014/122184A1. In any of the following examples, the deuterated catecholamine derivative, for example Composition 1, may be administered at a therapeutically effective dose or a sub-therapeutically effective amount. Examples of dosages include 17.5 mg, 20 mg, 25 mg, 35 mg, 40 mg, and 50 mg. Non-limiting examples of formulations are shown in the tables below.

Deuterated catecholamine derivatives such as Composition 1 may be formulated separately from, and then administered in combination with, opicapone.

### Formulation 1a-1h: Tablet with Film Coating Containing Composition 1

| Formulation | | | | | | | |
|---|---|---|---|---|---|---|---|
| 1a | 1b | 1c | 1d | 1e | 1f | 1g | 1h |

Composition of the core:

| | | | | | | | | | | | | | | | | |
|---|---|---|---|---|---|---|---|---|---|---|---|---|---|---|---|---|
| Composition 1 | 500.00 | mg | 300.00 | mg | 250.00 | mg | 150.00 | mg | 100.00 | mg | 60.00 | mg | 50.00 | mg | 30.00 | mg |
| Povidone | 20.00 | mg | 20.00 | mg | 20.00 | mg | 20.00 | mg | 20.00 | mg | 20.00 | mg | 20.00 | mg | 20.00 | mg |
| Sorbitol | 7.00 | mg | 7.00 | mg | 7.00 | mg | 7.00 | mg | 7.00 | mg | 7.00 | mg | 7.00 | mg | 7.00 | mg |
| Silicon dioxide, highly | 2 | mg | 2 | mg | 2 | mg | 2 | mg | 2 | mg | 2 | mg | 2 | mg | 2 | mg |
| dispersed | | | | | | | | | | | | | | | | |
| Pregelatinated starch | 40.00 | mg | 40.00 | mg | 40.00 | mg | 40.00 | mg | 40.00 | mg | 40.00 | mg | 40.00 | mg | 40.00 | mg |
| Crosscarmellose- | 13.30 | mg | 13.30 | mg | 13.30 | mg | 13.30 | mg | 13.30 | mg | 13.30 | mg | 13.30 | mg | 13.30 | mg |
| sodium | | | | | | | | | | | | | | | | |
| Carmellose sodium | 20.05 | mg | 20.05 | mg | 20.05 | mg | 20.05 | mg | 20.05 | mg | 20.05 | mg | 20.05 | mg | 20.05 | mg |
| Microcrystalline | 41.00 | mg | 41.00 | mg | 41.00 | mg | 41.00 | mg | 41.00 | mg | 41.00 | mg | 41.00 | mg | 41.00 | mg |
| cellulose | | | | | | | | | | | | | | | | |
| Magnesium stearate | 2.00 | mg | 2.00 | mg | 2.00 | mg | 2.00 | mg | 2.00 | mg | 2.00 | mg | 2.00 | mg | 2.00 | mg |

Film coating:

| | | | | | | | | | | | | | | | | |
|---|---|---|---|---|---|---|---|---|---|---|---|---|---|---|---|---|
| Hydroxypropylmethyl- | 16.00 | mg | 16.00 | mg | 16.00 | mg | 16.00 | mg | 16.00 | mg | 16.00 | mg | 16.00 | mg | 16.00 | mg |
| cellulose | | | | | | | | | | | | | | | | |
| Macrogol 400 ™ | 2.50 | mg | 2.50 | mg | 2.50 | mg | 2.50 | mg | 2.50 | mg | 2.50 | mg | 2.50 | mg | 2.50 | mg |
| Titanium oxide | 3.00 | mg | 3.00 | mg | 3.00 | mg | 3.00 | mg | 3.00 | mg | 3.00 | mg | 3.00 | mg | 3.00 | mg |
| Talc | 3.00 | mg | 3.00 | mg | 3.00 | mg | 3.00 | mg | 3.00 | mg | 3.00 | mg | 3.00 | mg | 3.00 | mg |

Preparation: Composition 1 and highly dispersed silicon dioxide are granulated in a compulsory mixer with a solution of povidone and sorbitol. The granules are dried, screened, mixed with pregelatinated starch, crosscarmellose sodium, carmellose sodium and microcrystalline cellulose, then combined with magnesium stearate and compressed into tablets. The tablets are film coated with hydroxypropylmethylcellulose, Macrogol, titanium dioxide and talc.

Deuterated catecholamine derivatives such as Composition 1 may be formulated with an AADCi such as carbidopa, but separately from, and then administered in combination with, opicapone.

### Formulation 2a-2f: Tablet with Film Coating Containing Composition 1 and Carbidopa

| Formulation | | | | | |
|---|---|---|---|---|---|
| 2a | 2b | 2c | 2d | 2e | 2f |

Composition of the core:

| | | | | | | | | | | | | |
|---|---|---|---|---|---|---|---|---|---|---|---|---|
| Composition 1 | 250.00 | mg | 150.00 | mg | 100.00 | mg | 60.00 | mg | 100.00 | mg | 60.00 | mg |
| Carbidopa | 50.00 | mg | 25.00 | mg | 25.00 | mg | 25.00 | mg | 50.00 | mg | 25.00 | mg |
| Povidone | 20.00 | mg | 20.00 | mg | 20.00 | mg | 20.00 | mg | 20.00 | mg | 20.00 | mg |
| Sorbitol | 7.00 | mg | 7.00 | mg | 7.00 | mg | 7.00 | mg | 7.00 | mg | 7.00 | mg |
| Silicon dioxide, highly | 2 | mg | 2 | mg | 2 | mg | 2 | mg | 2 | mg | 2 | mg |
| dispersed | | | | | | | | | | | | |
| Pregelatinated starch | 40.00 | mg | 40.00 | mg | 40.00 | mg | 40.00 | mg | 40.00 | mg | 40.00 | mg |
| Crosscarmellose-sodium | 13.30 | mg | 13.30 | mg | 13.30 | mg | 13.30 | mg | 13.30 | mg | 13.30 | mg |
| Carmellose sodium | 20.05 | mg | 20.05 | mg | 20.05 | mg | 20.05 | mg | 20.05 | mg | 20.05 | mg |
| Microcrystalline cellulose | 41.00 | mg | 41.00 | mg | 41.00 | mg | 41.00 | mg | 41.00 | mg | 41.00 | mg |
| Magnesium stearate | 2.00 | mg | 2.00 | mg | 2.00 | mg | 2.00 | mg | 2.00 | mg | 2.00 | mg |

Film coating:

| | | | | | | | | | | | | |
|---|---|---|---|---|---|---|---|---|---|---|---|---|
| Hydroxypropylmethylcellulose | 16.00 | mg | 16.00 | mg | 16.00 | mg | 16.00 | mg | 16.00 | mg | 16.00 | mg |
| Macrogol 400 ™ | 2.50 | mg | 2.50 | mg | 2.50 | mg | 2.50 | mg | 2.50 | mg | 2.50 | mg |
| Titanium oxide | 3.00 | mg | 3.00 | mg | 3.00 | mg | 3.00 | mg | 3.00 | mg | 3.00 | mg |
| Talc | 3.00 | mg | 3.00 | mg | 3.00 | mg | 3.00 | mg | 3.00 | mg | 3.00 | mg |

Preparation: Composition 1, carbidopa and highly dispersed silicon dioxide are granulated in a compulsory mixer with a solution of povidone and sorbitol. The granules are dried, screened, mixed with pregelatinated starch, crosscarmellose sodium, carmellose sodium and microcrystalline cellulose, then combined with magnesium stearate and compressed into tablets. The tablets are film coated with hydroxypropylmethylcellulose, Macrogol, titanium dioxide and talc.

### Formulation 3a-3f: Tablet with Film Coating Containing Microencapsulated Composition 1 and Carbidopa

| Formulation | | | | | |
|---|---|---|---|---|---|
| 3a | 3b | 3c | 3d | 3e | 3f |

Composition of the Core

| | | | | | | | | | | | | |
|---|---|---|---|---|---|---|---|---|---|---|---|---|
| Composition 1 | 250.00 | mg | 150.00 | mg | 100.00 | mg | 60.00 | mg | 100.00 | mg | 60.00 | mg |
| Carbidopa | 50.00 | mg | 50.00 | mg | 25.00 | mg | 25.00 | mg | 50.00 | mg | 10.00 | mg |
| Tartaric acid | 5.00 | mg | 5.00 | mg | 5.00 | mg | 5.00 | mg | 5.00 | mg | 5.00 | mg |
| Povidone | 20.00 | mg | 20.00 | mg | 20.00 | mg | 20.00 | mg | 20.00 | mg | 20.00 | mg |
| Sorbitol | 7.00 | mg | 7.00 | mg | 7.00 | mg | 7.00 | mg | 7.00 | mg | 7.00 | mg |
| EUDRAGIT ® RLTM solid | 20.00 | mg | 20.00 | mg | 20.00 | mg | 20.00 | mg | 20.00 | mg | 20.00 | mg |
| Silicon dioxide, highly | 2 | mg | 2 | mg | 2 | mg | 2 | mg | 2 | mg | 2 | mg |
| dispersed | | | | | | | | | | | | |
| Pregelatinated starch | 40.00 | mg | 40.00 | mg | 40.00 | mg | 40.00 | mg | 40.00 | mg | 40.00 | mg |
| Crosscarmellose-sodium | 13.30 | mg | 13.30 | mg | 13.30 | mg | 13.30 | mg | 13.30 | mg | 13.30 | mg |
| Carmellose sodium | 20.05 | mg | 20.05 | mg | 20.05 | mg | 20.05 | mg | 20.05 | mg | 20.05 | mg |
| Microcrystalline cellulose | 41.00 | mg | 41.00 | mg | 41.00 | mg | 41.00 | mg | 41.00 | mg | 41.00 | mg |
| Magnesium stearate | 2.00 | mg | 2.00 | mg | 2.00 | mg | 2.00 | mg | 2.00 | mg | 2.00 | mg |

Film coating:

| | | | | | | | | | | | | |
|---|---|---|---|---|---|---|---|---|---|---|---|---|
| Hydroxypropylmethyl- | 16.00 | mg | 16.00 | mg | 16.00 | mg | 16.00 | mg | 16.00 | mg | 16.00 | mg |
| cellulose | | | | | | | | | | | | |
| Macrogol 400 ™ | 2.50 | mg | 2.50 | mg | 2.50 | mg | 2.50 | mg | 2.50 | mg | 2.50 | mg |
| Titanium oxide | 3.00 | mg | 3.00 | mg | 3.00 | mg | 3.00 | mg | 3.00 | mg | 3.00 | mg |
| Talc | 3.00 | mg | 3.00 | mg | 3.00 | mg | 3.00 | mg | 3.00 | mg | 3.00 | mg |

Preparation: Composition 1, Carbidopa, sorbitol and Eudragit are microencapsulated and homogenised in a barrel mixer with tartaric acid, highly dispersed silicon dioxide, povidone, pregelatinated starch, crosscarmellose sodium, carmellose sodium and microcrystalline cellulose, then combined with magnesium stearate and compressed into tablets. The tablets are film coated with hydroxypropylmethylcellulose, Macrogol, titanium dioxide and talc.

### Formulations 4a-4f: Tablet with Film Coating Containing Microencapsulated Composition 1 and Benserazide

| Formulation | | | | | |
|---|---|---|---|---|---|
| 4a | 4b | 4c | 4d | 4e | 4f |

Composition of the core:

| | | | | | | | | | | | | |
|---|---|---|---|---|---|---|---|---|---|---|---|---|
| Composition 1 | 200.00 | mg | 120.00 | mg | 100.00 | mg | 60.00 | mg | 50.00 | mg | 30.00 | mg |
| Benserazide | 50.00 | mg | 50.00 | mg | 25.00 | mg | 25.00 | mg | 12.50 | mg | 12.50 | mg |
| Tartaric acid | 5.00 | mg | 5.00 | mg | 5.00 | mg | 5.00 | mg | 5.00 | mg | 5.00 | mg |
| Povidone | 20.00 | mg | 20.00 | mg | 20.00 | mg | 20.00 | mg | 20.00 | mg | 20.00 | mg |
| Sorbitol | 7.00 | mg | 7.00 | mg | 7.00 | mg | 7.00 | mg | 7.00 | mg | 7.00 | mg |
| EUDRAGIT RL ® solid | 20.00 | mg | 20.00 | mg | 20.00 | mg | 20.00 | mg | 20.00 | mg | 20.00 | mg |
| Silicon dioxide, highly | 2 | mg | 2 | mg | 2 | mg | 2 | mg | 2 | mg | 2 | mg |
| dispersed | | | | | | | | | | | | |
| Pregelatinated starch | 40.00 | mg | 40.00 | mg | 40.00 | mg | 40.00 | mg | 40.00 | mg | 40.00 | mg |
| Crosscarmellose-sodium | 13.30 | mg | 13.30 | mg | 13.30 | mg | 13.30 | mg | 13.30 | mg | 13.30 | mg |
| Carmellose sodium | 20.05 | mg | 20.05 | mg | 20.05 | mg | 20.05 | mg | 20.05 | mg | 20.05 | mg |
| Microcrystalline cellulose | 41.00 | mg | 41.00 | mg | 41.00 | mg | 41.00 | mg | 41.00 | mg | 41.00 | mg |
| Magnesium stearate | 2.00 | mg | 2.00 | mg | 2.00 | mg | 2.00 | mg | 2.00 | mg | 2.00 | mg |

Film coating:

| | | | | | | | | | | | | |
|---|---|---|---|---|---|---|---|---|---|---|---|---|
| Hydroxypropylmethylcellulose | 16.00 | mg | 16.00 | mg | 16.00 | mg | 16.00 | mg | 16.00 | mg | 16.00 | mg |
| Macrogol 400 ™ | 2.50 | mg | 2.50 | mg | 2.50 | mg | 2.50 | mg | 2.50 | mg | 2.50 | mg |
| Titanium oxide | 3.00 | mg | 3.00 | mg | 3.00 | mg | 3.00 | mg | 3.00 | mg | 3.00 | mg |
| Talc | 3.00 | mg | 3.00 | mg | 3.00 | mg | 3.00 | mg | 3.00 | mg | 3.00 | mg |

Preparation: as given above for Formulation 2.

### Formulations 5a-5f: Tablet with Film Coating Containing Composition 1 and Benserazide

| Formulation | | | | | |
|---|---|---|---|---|---|
| 5a | 5b | 5c | 5d | 5e | 5f |

Composition of the core:

| | | | | | | | | | | | | |
|---|---|---|---|---|---|---|---|---|---|---|---|---|
| Composition 1 | 200.00 | mg | 120.00 | mg | 100.00 | mg | 60.00 | mg | 50.00 | mg | 30.00 | mg |
| Benserazide | 75.00 | mg | 50.00 | mg | 25.00 | mg | 25.00 | mg | 12.50 | mg | 12.50 | mg |
| Povidone | 20.00 | mg | 20.00 | mg | 20.00 | mg | 20.00 | mg | 20.00 | mg | 20.00 | mg |
| Sorbitol | 7.00 | mg | 7.00 | mg | 7.00 | mg | 7.00 | mg | 7.00 | mg | 7.00 | mg |
| Silicon dioxide, highly | 2 | mg | 2 | mg | 2 | mg | 2 | mg | 2 | mg | 2 | mg |
| dispersed | | | | | | | | | | | | |
| Pregelatinated starch | 40.00 | mg | 40.00 | mg | 40.00 | mg | 40.00 | mg | 40.00 | mg | 40.00 | mg |
| Crosscarmellose-sodium | 13.30 | mg | 13.30 | mg | 13.30 | mg | 13.30 | mg | 13.30 | mg | 13.30 | mg |
| Carmel lose-sodium | 20.05 | mg | 20.05 | mg | 20.05 | mg | 20.05 | mg | 20.05 | mg | 20.05 | mg |
| Microcrystalline cellulose | 41.00 | mg | 41.00 | mg | 41.00 | mg | 41.00 | mg | 41.00 | mg | 41.00 | mg |
| Magnesium stearate | 2.00 | mg | 2.00 | mg | 2.00 | mg | 2.00 | mg | 2.00 | mg | 2.00 | mg |

Film coating:

| | | | | | | | | | | | | |
|---|---|---|---|---|---|---|---|---|---|---|---|---|
| Hydroxypropylmethylcellulose | 16.00 | mg | 16.00 | mg | 16.00 | mg | 16.00 | mg | 16.00 | mg | 16.00 | mg |
| Macrogol 400 ™ | 2.50 | mg | 2.50 | mg | 2.50 | mg | 2.50 | mg | 2.50 | mg | 2.50 | mg |
| Titanium oxide | 3.00 | mg | 3.00 | mg | 3.00 | mg | 3.00 | mg | 3.00 | mg | 3.00 | mg |
| Talc | 3.00 | mg | 3.00 | mg | 3.00 | mg | 3.00 | mg | 3.00 | mg | 3.00 | mg |

Preparation: Composition 1, carbidopa, and highly dispersed silicon dioxide are granulated in a compulsory mixer with a solution of povidone and sorbitol. The granules are dried, screened, mixed with pregelatinated starch, crosscarmellose sodium, carmellose sodium and microcrystalline cellulose, then combined with magnesium stearate and compressed into tablets. The tablets are film coated with hydroxypropylmethylcellulose, Macrogol, titanium dioxide and talc.

The formulations above, and variations thereof, may be administered in combination with opicapone.

### Formulations of Opicapone

The following are examples of how opicapone may be formulated. See, e.g., US 2014/0045900 A1. In any of the following examples, opicapone may be administered at a therapeutically effective dose or a sub-therapeutically effective amount. Examples of dosages include 5, 15, 30, 25, and 50 mg.

### Formulation 6: Opicapone

| Ingredient | Percent |
|---|---|
| Opicapone | 15.00% |
| Lactose monohydrate | 43.00% |
| Microcrystalline cellulose | 30.00% |
| Povidone | 4.00% |
| Croscarmellose sodium | 5.00% |
| Talc | 2.00% |
| Magnesium stearate | 1.00% |

### Formulation 7: Opicapone

| Ingredient | Percent |
|---|---|
| Opicapone | 15.00% |
| Microcrystalline cellulose | 72.50% |
| Ethylcellulose | 5.00% |
| Sodium starch glycolate | 6.00% |
| Colloidal Silicon Dioxide | 0.50% |
| Magnesium stearate | 1.00% |

### Formulation 8: Opicapone

| Ingredient | Percent |
|---|---|
| Opicapone | 20.00% |
| Microcrystalline cellulose | 25.00% |
| Calcium Phosphate, dibasic | 40.00% |
| dihydrate | |
| Povidone | 6.00% |
| Croscarmellose sodium | 6.00% |
| Talc | 2.00% |
| Magnesium stearate | 1.00% |

Opicapone may thus be formulated separately from, and then administered in combination, with a deuterated catecholamine derivative, optionally together with an additional agent such as an AADCi.

### Combination Formulations of Deuterated Catecholamine Derivatives

Alternatively, deuterated catecholamine derivatives such as Composition 1 may be formulated together with opicapone and, optionally, an AADCi such as carbidopa or benserazide.

### Formulations 9a-9g, 10a-10g, 11a-11g, 12a-12g, 13a-13g, 14a-14g, 15a-15g, and 16a-16g: Tablets with Film Coatings Containing Composition 1 and Carbidopa and Opicapone

| Formulation No. | | | | | | |
|---|---|---|---|---|---|---|
| 9a | 9b | 9c | 9d | 9e | 9f | 9g |

Composition of the core:

| | | | | | | | | | | | | | | |
|---|---|---|---|---|---|---|---|---|---|---|---|---|---|---|
| Composition 1 | 25.00 | mg | 25.00 | mg | 25.00 | mg | 25.00 | mg | 25.00 | mg | 25.00 | mg | 25.00 | mg |
| Carbidopa | 5.00 | mg | 5.00 | mg | 5.00 | mg | 5.00 | mg | 5.00 | mg | 5.00 | mg | 5.00 | mg |
| Opicapone | 100.00 | mg | 50.00 | mg | 30.00 | mg | 25.00 | mg | 15.00 | mg | 10.00 | mg | 5.00 | mg |
| Povidon K30 | 20.00 | mg | 20.00 | mg | 20.00 | mg | 20.00 | mg | 20.00 | mg | 20.00 | mg | 20.00 | mg |
| Crospovidon Type B | 15.00 | mg | 15.00 | mg | 15.00 | mg | 15.00 | mg | 15.00 | mg | 15.00 | mg | 15.00 | mg |
| Mannitol | 9.00 | mg | 9.00 | mg | 9.00 | mg | 9.00 | mg | 9.00 | mg | 9.00 | mg | 9.00 | mg |
| Silicon dioxide, highly | 2 | mg | 2 | mg | 2 | mg | 2 | mg | 2 | mg | 2 | mg | 2 | mg |
| dispersed | | | | | | | | | | | | | | |
| Pregelatinated starch | 40.00 | mg | 40.00 | mg | 40.00 | mg | 40.00 | mg | 40.00 | mg | 40.00 | mg | 40.00 | mg |
| Crosscarmellose-sodium | 13.30 | mg | 13.30 | mg | 13.30 | mg | 13.30 | mg | 13.30 | mg | 13.30 | mg | 13.30 | mg |
| Carmellose-sodium | 20.05 | mg | 20.05 | mg | 20.05 | mg | 20.05 | mg | 20.05 | mg | 20.05 | mg | 20.05 | mg |
| Microcrystalline cellulose | 41.00 | mg | 41.00 | mg | 41.00 | mg | 41.00 | mg | 41.00 | mg | 41.00 | mg | 41.00 | mg |
| Magnesium stearate | 2.00 | mg | 2.00 | mg | 2.00 | mg | 2.00 | mg | 2.00 | mg | 2.00 | mg | 2.00 | mg |

Film coating:

| | | | | | | | | | | | | | | |
|---|---|---|---|---|---|---|---|---|---|---|---|---|---|---|
| Hydroxypropylmethylcellulose | 16.0 | mg | 16.0 | mg | 16.0 | mg | 16.0 | mg | 16.0 | mg | 16.0 | mg | 16.0 | mg |
| Macrogol 400 TM | 2.50 | mg | 2.50 | mg | 2.50 | mg | 2.50 | mg | 2.50 | mg | 2.50 | mg | 2.50 | mg |
| Titanium oxide | 3.0 | mg | 3.0 | mg | 3.0 | mg | 3.0 | mg | 3.0 | mg | 3.0 | mg | 3.0 | mg |
| Talc | 3.0 | mg | 3.0 | mg | 3.0 | mg | 3.0 | mg | 3.0 | mg | 3.0 | mg | 3.0 | mg |

| Formulation No. | | | | | | |
|---|---|---|---|---|---|---|
| 10a | 10b | 10c | 10d | 10e | 10f | 10g |

Composition of the core:

| | | | | | | | | | | | | | | |
|---|---|---|---|---|---|---|---|---|---|---|---|---|---|---|
| Composition 1 | 60.00 | mg | 60.00 | mg | 60.00 | mg | 60.00 | mg | 60.00 | mg | 60.00 | mg | 60.00 | mg |
| Carbidopa | 50.00 | mg | 50.00 | mg | 25.00 | mg | 25.00 | mg | 25.00 | mg | 25.00 | mg | 10.00 | mg |
| Opicapone | 100.00 | mg | 50.00 | mg | 30.00 | mg | 25.00 | mg | 15.00 | mg | 10.00 | mg | 5.00 | mg |
| Povidon K30 | 20.00 | mg | 20.00 | mg | 20.00 | mg | 20.00 | mg | 20.00 | mg | 20.00 | mg | 20.00 | mg |
| Crospovidon Type B | 15.00 | mg | 15.00 | mg | 15.00 | mg | 15.00 | mg | 15.00 | mg | 15.00 | mg | 15.00 | mg |
| Mannitol | 9.00 | mg | 9.00 | mg | 9.00 | mg | 9.00 | mg | 9.00 | mg | 9.00 | mg | 9.00 | mg |
| Silicon dioxide, highly | 2 | mg | 2 | mg | 2 | mg | 2 | mg | 2 | mg | 2 | mg | 2 | mg |
| dispersed | | | | | | | | | | | | | | |
| Pregelatinated starch | 40.00 | mg | 40.00 | mg | 40.00 | mg | 40.00 | mg | 40.00 | mg | 40.00 | mg | 40.00 | mg |
| Crosscarmellose-sodium | 13.30 | mg | 13.30 | mg | 13.30 | mg | 13.30 | mg | 13.30 | mg | 13.30 | mg | 13.30 | mg |
| Carmellose-sodium | 20.05 | mg | 20.05 | mg | 20.05 | mg | 20.05 | mg | 20.05 | mg | 20.05 | mg | 20.05 | mg |
| Microcrystalline cellulose | 41.00 | mg | 41.00 | mg | 41.00 | mg | 41.00 | mg | 41.00 | mg | 41.00 | mg | 41.00 | mg |
| Magnesium stearate | 2.00 | mg | 2.00 | mg | 2.00 | mg | 2.00 | mg | 2.00 | mg | 2.00 | mg | 2.00 | mg |

Film coating:

| | | | | | | | | | | | | | | |
|---|---|---|---|---|---|---|---|---|---|---|---|---|---|---|
| Hydroxypropylmethylcellulose | 16.0 | mg | 16.0 | mg | 16.0 | mg | 16.0 | mg | 16.0 | mg | 16.0 | mg | 16.0 | mg |
| Macrogol 400 TM | 2.50 | mg | 2.50 | mg | 2.50 | mg | 2.50 | mg | 2.50 | mg | 2.50 | mg | 2.50 | mg |
| Titanium oxide | 3.0 | mg | 3.0 | mg | 3.0 | mg | 3.0 | mg | 3.0 | mg | 3.0 | mg | 3.0 | mg |
| Talc | 3.0 | mg | 3.0 | mg | 3.0 | mg | 3.0 | mg | 3.0 | mg | 3.0 | mg | 3.0 | mg |

| Formulation No. | | | | | | |
|---|---|---|---|---|---|---|
| 11a | 11b | 11c | 11d | 11e | 11f | 11g |

Composition of the core:

| | | | | | | | | | | | | | | |
|---|---|---|---|---|---|---|---|---|---|---|---|---|---|---|
| Composition 1 | 100.00 | mg | 100.00 | mg | 100.00 | mg | 100.00 | mg | 100.00 | mg | 100.00 | mg | 100.00 | mg |
| Carbidopa | 50.00 | mg | 50.00 | mg | 25.00 | mg | 25.00 | mg | 25.00 | mg | 25.00 | mg | 10.00 | mg |
| Opicapone | 100.00 | mg | 50.00 | mg | 30.00 | mg | 25.00 | mg | 15.00 | mg | 10.00 | mg | 5.00 | mg |
| Povidon K30 | 20.00 | mg | 20.00 | mg | 20.00 | mg | 20.00 | mg | 20.00 | mg | 20.00 | mg | 20.00 | mg |
| Crospovidon Type B | 15.00 | mg | 15.00 | mg | 15.00 | mg | 15.00 | mg | 15.00 | mg | 15.00 | mg | 15.00 | mg |
| Mannitol | 9.00 | mg | 9.00 | mg | 9.00 | mg | 9.00 | mg | 9.00 | mg | 9.00 | mg | 9.00 | mg |
| Silicon dioxide, highly | 2 | mg | 2 | mg | 2 | mg | 2 | mg | 2 | mg | 2 | mg | 2 | mg |
| dispersed | | | | | | | | | | | | | | |
| Pregelatinated starch | 40.00 | mg | 40.00 | mg | 40.00 | mg | 40.00 | mg | 40.00 | mg | 40.00 | mg | 40.00 | mg |
| Crosscarmellose-sodium | 13.30 | mg | 13.30 | mg | 13.30 | mg | 13.30 | mg | 13.30 | mg | 13.30 | mg | 13.30 | mg |
| Carmellose-sodium | 20.05 | mg | 20.05 | mg | 20.05 | mg | 20.05 | mg | 20.05 | mg | 20.05 | mg | 20.05 | mg |
| Microcrystalline cellulose | 41.00 | mg | 41.00 | mg | 41.00 | mg | 41.00 | mg | 41.00 | mg | 41.00 | mg | 41.00 | mg |
| Magnesium stearate | 2.00 | mg | 2.00 | mg | 2.00 | mg | 2.00 | mg | 2.00 | mg | 2.00 | mg | 2.00 | mg |

Film coating:

| | | | | | | | | | | | | | | |
|---|---|---|---|---|---|---|---|---|---|---|---|---|---|---|
| Hydroxypropylmethylcellulose | 16.0 | mg | 16.0 | mg | 16.0 | mg | 16.0 | mg | 16.0 | mg | 16.0 | mg | 16.0 | mg |
| Macrogol 400 TM | 2.50 | mg | 2.50 | mg | 2.50 | mg | 2.50 | mg | 2.50 | mg | 2.50 | mg | 2.50 | mg |
| Titanium oxide | 3.0 | mg | 3.0 | mg | 3.0 | mg | 3.0 | mg | 3.0 | mg | 3.0 | mg | 3.0 | mg |
| Talc | 3.0 | mg | 3.0 | mg | 3.0 | mg | 3.0 | mg | 3.0 | mg | 3.0 | mg | 3.0 | mg |

| Formulation No. | | | | | | |
|---|---|---|---|---|---|---|
| 12a | 12b | 12c | 12d | 12e | 12f | 12g |

Composition of the core:

| | | | | | | | | | | | | | | |
|---|---|---|---|---|---|---|---|---|---|---|---|---|---|---|
| Composition 1 | 60.00 | mg | 60.00 | mg | 60.00 | mg | 60.00 | mg | 60.00 | mg | 60.00 | mg | 60.00 | mg |
| Carbidopa | 50.00 | mg | 25.00 | mg | 25.00 | mg | 25.00 | mg | 25.00 | mg | 25.00 | mg | 25.00 | mg |
| Opicapone | 100.00 | mg | 50.00 | mg | 30.00 | mg | 25.00 | mg | 15.00 | mg | 10.00 | mg | 5.00 | mg |
| Povidon K30 | 20.00 | mg | 20.00 | mg | 20.00 | mg | 20.00 | mg | 20.00 | mg | 20.00 | mg | 20.00 | mg |
| Crospovidon Type B | 15.00 | mg | 15.00 | mg | 15.00 | mg | 15.00 | mg | 15.00 | mg | 15.00 | mg | 15.00 | mg |
| Mannitol | 9.00 | mg | 9.00 | mg | 9.00 | mg | 9.00 | mg | 9.00 | mg | 9.00 | mg | 9.00 | mg |
| Silicon dioxide, highly | 2 | mg | 2 | mg | 2 | mg | 2 | mg | 2 | mg | 2 | mg | 2 | mg |
| dispersed | | | | | | | | | | | | | | |
| Pregelatinated starch | 40.00 | mg | 40.00 | mg | 40.00 | mg | 40.00 | mg | 40.00 | mg | 40.00 | mg | 40.00 | mg |
| Crosscarmellose-sodium | 13.30 | mg | 13.30 | mg | 13.30 | mg | 13.30 | mg | 13.30 | mg | 13.30 | mg | 13.30 | mg |
| Carmellose-sodium | 20.05 | mg | 20.05 | mg | 20.05 | mg | 20.05 | mg | 20.05 | mg | 20.05 | mg | 20.05 | mg |
| Microcrystalline cellulose | 41.00 | mg | 41.00 | mg | 41.00 | mg | 41.00 | mg | 41.00 | mg | 41.00 | mg | 41.00 | mg |
| Magnesium stearate | 2.00 | mg | 2.00 | mg | 2.00 | mg | 2.00 | mg | 2.00 | mg | 2.00 | mg | 2.00 | mg |

Film coating:

| | | | | | | | | | | | | | | |
|---|---|---|---|---|---|---|---|---|---|---|---|---|---|---|
| Hydroxypropylmethylcellulose | 16.0 | mg | 16.0 | mg | 16.0 | mg | 16.0 | mg | 16.0 | mg | 16.0 | mg | 16.0 | mg |
| Macrogol 400 TM | 2.50 | mg | 2.50 | mg | 2.50 | mg | 2.50 | mg | 2.50 | mg | 2.50 | mg | 2.50 | mg |
| Titanium oxide | 3.0 | mg | 3.0 | mg | 3.0 | mg | 3.0 | mg | 3.0 | mg | 3.0 | mg | 3.0 | mg |
| Talc | 3.0 | mg | 3.0 | mg | 3.0 | mg | 3.0 | mg | 3.0 | mg | 3.0 | mg | 3.0 | mg |

| Formulation No. | | | | | | |
|---|---|---|---|---|---|---|
| 13a | 13b | 13c | 13d | 13e | 13f | 13g |

Composition of the core:

| | | | | | | | | | | | | | | |
|---|---|---|---|---|---|---|---|---|---|---|---|---|---|---|
| Composition 1 | 100.00 | mg | 100.00 | mg | 100.00 | mg | 100.00 | mg | 100.00 | mg | 100.00 | mg | 100.00 | mg |
| Carbidopa | 25.00 | mg | 25.00 | mg | 25.00 | mg | 25.00 | mg | 25.00 | mg | 25.00 | mg | 25.00 | mg |
| Opicapone | 100.00 | mg | 50.00 | mg | 30.00 | mg | 25.00 | mg | 15.00 | mg | 10.00 | mg | 5.00 | mg |
| Povidon K30 | 20.00 | mg | 20.00 | mg | 20.00 | mg | 20.00 | mg | 20.00 | mg | 20.00 | mg | 20.00 | mg |
| Crospovidon Type B | 15.00 | mg | 15.00 | mg | 15.00 | mg | 15.00 | mg | 15.00 | mg | 15.00 | mg | 15.00 | mg |
| Mannitol | 9.00 | mg | 9.00 | mg | 9.00 | mg | 9.00 | mg | 9.00 | mg | 9.00 | mg | 9.00 | mg |
| Silicon dioxide, highly | 2 | mg | 2 | mg | 2 | mg | 2 | mg | 2 | mg | 2 | mg | 2 | mg |
| dispersed | | | | | | | | | | | | | | |
| Pregelatinated starch | 40.00 | mg | 40.00 | mg | 40.00 | mg | 40.00 | mg | 40.00 | mg | 40.00 | mg | 40.00 | mg |
| Crosscarmellose-sodium | 13.30 | mg | 13.30 | mg | 13.30 | mg | 13.30 | mg | 13.30 | mg | 13.30 | mg | 13.30 | mg |
| Carmellose-sodium | 20.05 | mg | 20.05 | mg | 20.05 | mg | 20.05 | mg | 20.05 | mg | 20.05 | mg | 20.05 | mg |
| Microcrystalline cellulose | 41.00 | mg | 41.00 | mg | 41.00 | mg | 41.00 | mg | 41.00 | mg | 41.00 | mg | 41.00 | mg |
| Magnesium stearate | 2.00 | mg | 2.00 | mg | 2.00 | mg | 2.00 | mg | 2.00 | mg | 2.00 | mg | 2.00 | mg |

Film coating:

| | | | | | | | | | | | | | | |
|---|---|---|---|---|---|---|---|---|---|---|---|---|---|---|
| Hydroxypropylmethylcellulose | 16.0 | mg | 16.0 | mg | 16.0 | mg | 16.0 | mg | 16.0 | mg | 16.0 | mg | 16.0 | mg |
| Macrogol 400 TM | 2.50 | mg | 2.50 | mg | 2.50 | mg | 2.50 | mg | 2.50 | mg | 2.50 | mg | 2.50 | mg |
| Titanium oxide | 3.0 | mg | 3.0 | mg | 3.0 | mg | 3.0 | mg | 3.0 | mg | 3.0 | mg | 3.0 | mg |
| Talc | 3.0 | mg | 3.0 | mg | 3.0 | mg | 3.0 | mg | 3.0 | mg | 3.0 | mg | 3.0 | mg |

| Formulation No. | | | | | | |
|---|---|---|---|---|---|---|
| 14a | 14b | 14c | 14d | 14e | 14f | 14g |

Composition of the core:

| | | | | | | | | | | | | | | |
|---|---|---|---|---|---|---|---|---|---|---|---|---|---|---|
| Composition 1 | 120.00 | mg | 120.00 | mg | 120.00 | mg | 120.00 | mg | 120.00 | mg | 120.00 | mg | 120.00 | mg |
| Carbidopa | 50.00 | mg | 50.00 | mg | 25.00 | mg | 25.00 | mg | 25.00 | mg | 25.00 | mg | 25.00 | mg |
| Opicapone | 100.00 | mg | 50.00 | mg | 30.00 | mg | 25.00 | mg | 15.00 | mg | 10.00 | mg | 5.00 | mg |
| Povidon K30 | 20.00 | mg | 20.00 | mg | 20.00 | mg | 20.00 | mg | 20.00 | mg | 20.00 | mg | 20.00 | mg |
| Crospovidon Type B | 15.00 | mg | 15.00 | mg | 15.00 | mg | 15.00 | mg | 15.00 | mg | 15.00 | mg | 15.00 | mg |
| Mannitol | 9.00 | mg | 9.00 | mg | 9.00 | mg | 9.00 | mg | 9.00 | mg | 9.00 | mg | 9.00 | mg |
| Silicon dioxide, highly | 2 | mg | 2 | mg | 2 | mg | 2 | mg | 2 | mg | 2 | mg | 2 | mg |
| dispersed | | | | | | | | | | | | | | |
| Pregelatinated starch | 40.00 | mg | 40.00 | mg | 40.00 | mg | 40.00 | mg | 40.00 | mg | 40.00 | mg | 40.00 | mg |
| Crosscarmellose-sodium | 13.30 | mg | 13.30 | mg | 13.30 | mg | 13.30 | mg | 13.30 | mg | 13.30 | mg | 13.30 | mg |
| Carmellose-sodium | 20.05 | mg | 20.05 | mg | 20.05 | mg | 20.05 | mg | 20.05 | mg | 20.05 | mg | 20.05 | mg |
| Microcrystalline cellulose | 41.00 | mg | 41.00 | mg | 41.00 | mg | 41.00 | mg | 41.00 | mg | 41.00 | mg | 41.00 | mg |
| Magnesium stearate | 2.00 | mg | 2.00 | mg | 2.00 | mg | 2.00 | mg | 2.00 | mg | 2.00 | mg | 2.00 | mg |

Film coating:

| | | | | | | | | | | | | | | |
|---|---|---|---|---|---|---|---|---|---|---|---|---|---|---|
| Hydroxypropylmethylcellulose | 16.0 | mg | 16.0 | mg | 16.0 | mg | 16.0 | mg | 16.0 | mg | 16.0 | mg | 16.0 | mg |
| Macrogol 400 TM | 2.50 | mg | 2.50 | mg | 2.50 | mg | 2.50 | mg | 2.50 | mg | 2.50 | mg | 2.50 | mg |
| Titanium oxide | 3.0 | mg | 3.0 | mg | 3.0 | mg | 3.0 | mg | 3.0 | mg | 3.0 | mg | 3.0 | mg |
| Talc | 3.0 | mg | 3.0 | mg | 3.0 | mg | 3.0 | mg | 3.0 | mg | 3.0 | mg | 3.0 | mg |

| Formulation No. | | | | | | |
|---|---|---|---|---|---|---|
| 15a | 15b | 15c | 15d | 15e | 15f | 15g |

Composition of the core:

| | | | | | | | | | | | | | | |
|---|---|---|---|---|---|---|---|---|---|---|---|---|---|---|
| Composition 1 | 150.00 | mg | 150.00 | mg | 150.00 | mg | 150.00 | mg | 150.00 | mg | 150.00 | mg | 150.00 | mg |
| Carbidopa | 50.00 | mg | 50.00 | mg | 25.00 | mg | 25.00 | mg | 25.00 | mg | 25.00 | mg | 25.00 | mg |
| Opicapone | 100.00 | mg | 50.00 | mg | 30.00 | mg | 25.00 | mg | 15.00 | mg | 10.00 | mg | 5.00 | mg |
| Povidon K30 | 20.00 | mg | 20.00 | mg | 20.00 | mg | 20.00 | mg | 20.00 | mg | 20.00 | mg | 20.00 | mg |
| Crospovidon Type B | 15.00 | mg | 15.00 | mg | 15.00 | mg | 15.00 | mg | 15.00 | mg | 15.00 | mg | 15.00 | mg |
| Mannitol | 9.00 | mg | 9.00 | mg | 9.00 | mg | 9.00 | mg | 9.00 | mg | 9.00 | mg | 9.00 | mg |
| Silicon dioxide, highly | 2 | mg | 2 | mg | 2 | mg | 2 | mg | 2 | mg | 2 | mg | 2 | mg |
| dispersed | | | | | | | | | | | | | | |
| Pregelatinated starch | 40.00 | mg | 40.00 | mg | 40.00 | mg | 40.00 | mg | 40.00 | mg | 40.00 | mg | 40.00 | mg |
| Crosscarmellose-sodium | 13.30 | mg | 13.30 | mg | 13.30 | mg | 13.30 | mg | 13.30 | mg | 13.30 | mg | 13.30 | mg |
| Carmellose-sodium | 20.05 | mg | 20.05 | mg | 20.05 | mg | 20.05 | mg | 20.05 | mg | 20.05 | mg | 20.05 | mg |
| Microcrystalline cellulose | 41.00 | mg | 41.00 | mg | 41.00 | mg | 41.00 | mg | 41.00 | mg | 41.00 | mg | 41.00 | mg |
| Magnesium stearate | 2.00 | mg | 2.00 | mg | 2.00 | mg | 2.00 | mg | 2.00 | mg | 2.00 | mg | 2.00 | mg |

Film coating:

| | | | | | | | | | | | | | | |
|---|---|---|---|---|---|---|---|---|---|---|---|---|---|---|
| Hydroxypropylmethylcellulose | 16.0 | mg | 16.0 | mg | 16.0 | mg | 16.0 | mg | 16.0 | mg | 16.0 | mg | 16.0 | mg |
| Macrogol 400 TM | 2.50 | mg | 2.50 | mg | 2.50 | mg | 2.50 | mg | 2.50 | mg | 2.50 | mg | 2.50 | mg |
| Titanium oxide | 3.0 | mg | 3.0 | mg | 3.0 | mg | 3.0 | mg | 3.0 | mg | 3.0 | mg | 3.0 | mg |
| Talc | 3.0 | mg | 3.0 | mg | 3.0 | mg | 3.0 | mg | 3.0 | mg | 3.0 | mg | 3.0 | mg |

| Formulation No. | | | | | | |
|---|---|---|---|---|---|---|
| 16a | 16b | 16c | 16d | 16e | 16f | 16g |

Composition of the core:

| | | | | | | | | | | | | | | |
|---|---|---|---|---|---|---|---|---|---|---|---|---|---|---|
| Composition 1 | 200.00 | mg | 200.00 | mg | 200.00 | mg | 200.00 | mg | 200.00 | mg | 200.00 | mg | 200.00 | mg |
| Carbidopa | 50.00 | mg | 50.00 | mg | 50.00 | mg | 50.00 | mg | 50.00 | mg | 50.00 | mg | 50.00 | mg |
| Opicapone | 100.00 | mg | 50.00 | mg | 30.00 | mg | 25.00 | mg | 15.00 | mg | 10.00 | mg | 5.00 | mg |
| Povidon K30 | 20.00 | mg | 20.00 | mg | 20.00 | mg | 20.00 | mg | 20.00 | mg | 20.00 | mg | 20.00 | mg |
| Crospovidon Type B | 15.00 | mg | 15.00 | mg | 15.00 | mg | 15.00 | mg | 15.00 | mg | 15.00 | mg | 15.00 | mg |
| Mannitol | 9.00 | mg | 9.00 | mg | 9.00 | mg | 9.00 | mg | 9.00 | mg | 9.00 | mg | 9.00 | mg |
| Silicon dioxide, highly | 2 | mg | 2 | mg | 2 | mg | 2 | mg | 2 | mg | 2 | mg | 2 | mg |
| dispersed | | | | | | | | | | | | | | |
| Pregelatinated starch | 40.00 | mg | 40.00 | mg | 40.00 | mg | 40.00 | mg | 40.00 | mg | 40.00 | mg | 40.00 | mg |
| Crosscarmellose-sodium | 13.30 | mg | 13.30 | mg | 13.30 | mg | 13.30 | mg | 13.30 | mg | 13.30 | mg | 13.30 | mg |
| Carmellose-sodium | 20.05 | mg | 20.05 | mg | 20.05 | mg | 20.05 | mg | 20.05 | mg | 20.05 | mg | 20.05 | mg |
| Microcrystalline cellulose | 41.00 | mg | 41.00 | mg | 41.00 | mg | 41.00 | mg | 41.00 | mg | 41.00 | mg | 41.00 | mg |
| Magnesium stearate | 2.00 | mg | 2.00 | mg | 2.00 | mg | 2.00 | mg | 2.00 | mg | 2.00 | mg | 2.00 | mg |

Film coating:

| | | | | | | | | | | | | | | |
|---|---|---|---|---|---|---|---|---|---|---|---|---|---|---|
| Hydroxypropylmethylcellulose | 16.0 | mg | 16.0 | mg | 16.0 | mg | 16.0 | mg | 16.0 | mg | 16.0 | mg | 16.0 | mg |
| Macrogol 400 TM | 2.50 | mg | 2.50 | mg | 2.50 | mg | 2.50 | mg | 2.50 | mg | 2.50 | mg | 2.50 | mg |
| Titanium oxide | 3.0 | mg | 3.0 | mg | 3.0 | mg | 3.0 | mg | 3.0 | mg | 3.0 | mg | 3.0 | mg |
| Talc | 3.0 | mg | 3.0 | mg | 3.0 | mg | 3.0 | mg | 3.0 | mg | 3.0 | mg | 3.0 | mg |

Preparation of the film coated tablets is as given above for Formulation 1.

### Biological Activity Assays

### Microdialysis in Sprague-Dawley Rats:

Female Sprague-Dawley rats weighing approximately 225 g were housed on a 12-hour light/dark cycle and kept on standard laboratory diet and water ad libitum. Microdialysis probes were implanted into the striatum prior to the experiment. During the experiment a physiological buffer solution was constantly pumped through the microdialysis probe and collected in fractions. The concentration of dopamine and other catecholamine derivatives in these samples was measured with HPLC and electrochemical detection (classical striatal microdialysis model). The application of conventional levodopa (plus carbidopa) leads to an increase of dopamine in the extracellular space in the striatal brain tissue. Deuterated levodopa as described above (plus carbidopa) leads to a prolonged half life of dopamine (about doubled) and increased central bioavailability (as described in previous patent application). Results from an experiment run similarly to this are shown in FIG. 1. Additional pretreatment of the rats with opicapone 2 mg/kg is expected to further increase the central half life of dopamine and lead to an even higher central bioavailability and “smoothened” dopamine levels (methods according to Bonifacio et al. (2015) See comment in PubMed Commons below Br J Pharmacol. 172(7):1739-52).

### Composition 1+Opicapone/Entacapone

To determine whether SD-1077 combined with Opicapone is superior to SD-1077 with entacapone, doses of entacapone and opicapone that produce a comparable degree of peripheral COMT inhibition were used, so that differences in their ability to enhance DA levels could be attributed to inhibition of central COMT and not to a higher supply of L-DOPA to the brain.

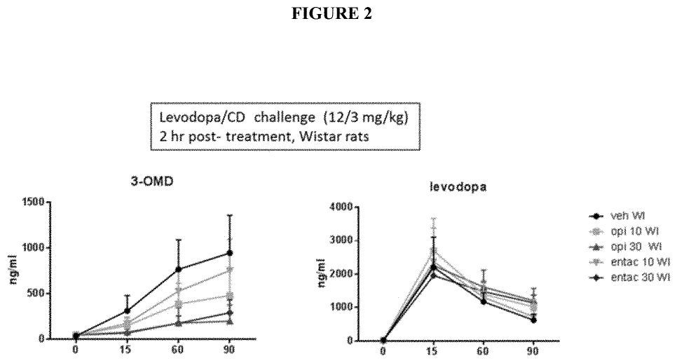

A single oral dose of levodopa+carbidopa was administered to adult male rats, following 2 hr pretreatment with an oral dose of vehicle or a single oral dose of opicapone or entacapone (30 mg/kg) The pharmacodynamic endpoint was the reduction of plasma concentrations of the COMT-derived metabolite 3-OMD. The findings show that opicapone and entacapone exerted a maximal effect on plasma levels of 3-OMD in Wistar rats at a dose of 30 mg/kg. See FIG. 2.

### Rodent Model of Parkinsonian Motor Performance and Dyskinesia

Female Sprague-Dawley rats weighing approximately 225 g are housed on a 12-hour light/dark cycle and kept on standard laboratory diet and water ad libitum. The rats are lesioned by unilateral injection of the neurotoxin 6-OHDA. The lesion is validated by measuring the rotational activity after i.p. injection of 2.5 mg/kg D-amphetamine. The anti-Parkinson effect (effect on motor performance) is evaluated by measurement of drug induced contralateral rotations. A dose effect is established to determine the equipotent (equi-effective) dose between i) conventional levodopa ii) conventional levodopa plus opicapone iii) deuterated levodopa (Composition 1), and iv) deuterated levodopa plus opicapone. Dyskinesia is evaluated after repeated treatment by scoring the animals for abnormal involuntary movements. The rats are scored by an observer blinded to the experimental design for limb, axial, and orolingual involuntary movements. The motor effect as a percentage of the effect caused by the control compound L-DOPA, the equipotent dose as percent of L-DOPA dose that caused the same effect on motor performance, are reported. As disclosed in WO2014/122184A1, Composition 1 (α,β,β*-D3-L-DOPA), α,β,β-D3-L-DOPA, and α,β,-D2-L-DOPA are all as effective as L-DOPA at lower doses, and Composition 1 (deuterated levodopa plus opicapone) demonstrated the lowest equivalent dose are compared to L-DOPA, and deuterated levodopa.

### 6-Hydroxydopamine Model of Parkinsonian Motor Performance in Wistar Rats

CD/Wistar rats weighing approximately 225 g were housed at a standard temperature (22±1° C.) and in a light-controlled environment (lights on from 7 am to 8 pm) with ad libitum access to food and water. The rats were lesioned by unilateral injection of the neurotoxin 6-OHDA into the medial forebrain bundle. After the 6-OHDA lesioning surgeries, the animals have a lesion maturation period of 14 days. On study day 15 after lesioning apomorphine (0.5 mg/kg s.c.) rotation asymmetry test for 60 minutes was performed to verify the success of the lesion.

On day 17 and after apomorphine screen rats were screened with L-Dopa 50 mg/kg+carbidopa 25 mg/kg (p.o.) in which rotational activity is measured for 90 min. The anti-Parkinson effect (effect on motor performance) was evaluated by measurement of drug induced contralateral rotations.

### Experimental Design

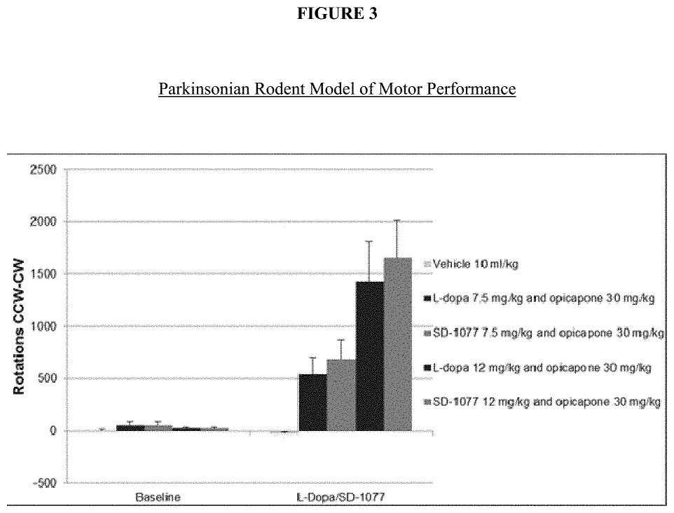

Cohort 1: 16 rats treated with Vehicle/L-Dopa/SD-1077 (7.5 mg/kg; 10 ml/kg) with catechol-O-methyltransferase (COMT) inhibitor (COMTi) opicapone
Cohort 2: 16 rats treated with Vehicle/L-Dopa/SD-1077 (12 mg/kg; 10 ml/kg) with COMTi opicapone
Cohort 3: 16 rats treated with Vehicle/Vehicle/Vehicle (10 ml/kg) with COMTi opicapone
Rats were dosed with vehicle or COMTi (opicapone) 30 mg/kg for 120 min, followed by administration of either vehicle or L-Dopa or SD-1077 in cross over dosing. After each dosing-testing round a washout period of 1 week was applied between the test articles. See FIG. 3.

Rotation Asymmetry Testing

Rats were fasted overnight before dosing and subsequent testing in asymmetry test. Rats were monitored for rotational asymmetry in automated rotometer bowls (TSE Systems, Germany) immediately after dosing is completed. Monitoring system was set to monitor full rotations) (360° and data were collected in 1 and 10 min bins for apomorphine and L-Dopa 50 mg/kg screen and in 10 min bins for the compound testing for 240 min (4 h) during the test. In the tests when COMT inhibitor opicapone is used, rotational activity was monitored for 15 min before and 120 min after opicapone administration, followed by 240 min test after delivery of L-Dopa or SD-1077. The rotation asymmetry score for each test was expressed as subtraction of the clockwise (CW) from counterclockwise (CCW) rotations. Each test session was performed in groups with maximum size 32 rats in a single rotometer set up.

### Microdialysis in Wistar Rats

This study examined the pharmacodynamic effects of Composition 1 (50 mg/kg, SD-1077) in conjunction with carbidopa (25 mg/kg) following COMTi treatment, on extracellular levels of L-DOPA (levodopa), 3-OMD (3-O-methyldopa), DA (dopamine), NE (norepinephrine), 3-MT (3-methoxytyramine) and DOPAC (3,4-dihydroxyphenylacetic acid), as well as their labeled equivalents when applicable, with simultaneous LMA (locomotor activity) assessment.

To this end, I-shaped probes (polyacrylonitrile membrane, BrainLink, the Netherlands) were implanted in the striatum (STR) of the animal. The probes were perfused with aCSF. Microdialysate samples were collected for 1 hour before dosing the animals with COMT treatment or vehicle. Two hours later, animals were treated with a cassette treatment of carbidopa with L-DOPA or Composition 1. After the second compound administration, microdialysate samples were collected for an additional 6 hours. LMA was recorded throughout the course of the microdialysis experiment, using a San Diego Instruments Photobeam Activity System-Home Cage (PAS-HC, San Diego, Calif.). In the dialysate samples, levels of L-DOPA, DA, DOPAC, HVA, and 3-MT, as well as their labeled equivalents when applicable, were quantified by LC-MS/MS.

Adult male Wistar rats (200-300 g) were used in the study, grouped as follows:

Treatment Groups

| Group | Treatment 1 (PO) | Treatment 2 (PO) | n |
|---|---|---|---|
| 1 | Vehicle | Composition 1 + Carbidopa | 6 |
| 3 | Opicapone (30 mg/kg) | Composition 1 + Carbidopa | 6 |
| 5 | Entacapone (30 mg/kg) | Composition 1 + Carbidopa | 6 |

### Dosing

| | dose | | Time | injection | |
|---|---|---|---|---|---|
| substance | (mg/kg) | cf | (hr) | route | volume |
| Vehicle | N/A | N/A | t = 0 | PO | 4 mL/kg |
| opicapone | 30 mg/kg | | t = 0 | PO | 4 mL/kg |
| entacapone | 30 mg/kg | | t = 0 | PO | 4 mL/kg |
| Composition 1 | 50 mg/kg | | t = 2 | PO | 4 mL/kg |
| carbidopa | 25 mg/kg | | | | |

Collected 2 × 50 μL aliquots of each unique dosing solution. Stored at −80 ° C.

The 30 mg/kg dose of opicapone and entacapone exerts a similar, maximal effect on plasma levels of 3-OMD in Wistar rats (FIG. 2)

Drug Administration

4 mL/kg of Vehicle, opicapone (30 mg/kg) or entacapone (30 mg/kg) was administered PO at t=0. Two hours later, vehicle (n=4; 4 mL/kg) or a 4 mL/kg cassette dose of Composition 1 (50 mg/kg)/carbidopa (25 mg/kg) or L-DOPA (50 mg/kg)/carbidopa was administered.

Microdialysis Procedure

Rats were anesthetized using isoflurane (2%, 800 mL/min O₂). Bupivacaine/epinephrine was used for local anesthesia and carprofen was used for peri-/post-operative analgesia. The animals were placed in a stereotaxic frame (Kopf instruments, USA). I-shaped microdialysis probes (polyacrylonitrile membrane, BrainLink, the Netherlands) were inserted into the STR (3 mm exposed surface). Coordinates for the tips of the probes in the STR were: posterior (AP)=+0.9 mm from bregma, lateral (L)=−3.0 mm to midline and ventral (V)=−6.5 mm to dura, the toothbar set at −3.3 mm. After surgery animals were kept individually in cages and provided food and water ad libitum.

Experiments were performed one day after surgery. On the day of the experiment, the probes of the animals were connected with flexible PEEK tubing to a microperfusion pump (Harvard PHD 2000 Syringe pump, Holliston, Mass. or similar). Microdialysis probes were perfused with aCSF containing 147 mM NaCl, 3.0 mM KCl, 1.2 mM CaCl₂) and 1.2 mM MgCl₂, at a flow rate of 1.5 μL/min. Microdialysis samples were collected for 30 minute periods by an automated fraction collector (820 Microsampler, Univentor, Malta or similar) into polystyrene mini-vials already containing 15 μL of 0.02M formic acid (FA) and 0.04% ascorbic acid in ultrapurified H₂O. Three basal samples were collected before the PO administration of 4 mL/kg of Vehicle, opicapone (30 mg/kg) or entacapone (30 mg/kg). Dialysates were collected for two hours before the administration of the second treatment (vehicle (n=4; 4 mL/kg) or 4 mL/kg cassette doses of Composition 1 (50 mg/kg)/carbidopa (25 mg/kg) or L-DOPA (50 mg/kg)/carbidopa). Samples were collected for an additional 6 hours following this second compound administration. All the dialysis samples were stored at −80° C. awaiting their analysis. After the experiment, the mice were sacrificed and brain tissue was collected for probe verification.

Locomotor Activity Assessment

Locomotor activity (LMA) was assessed using a San Diego Instruments Photobeam Activity System-Home Cage (PAS-HC, San Diego, Calif.). The PAS-HC system allows for detection of locomotor activity in an animal's home cage using a photobeam detection system. Distance traveled or locomotor activity was measured as a total number of beam breaks during an experimental session. All experiments were performed under normal lighting conditions. Locomotor activity was assessed during the entire course of microdialysis experimentation.

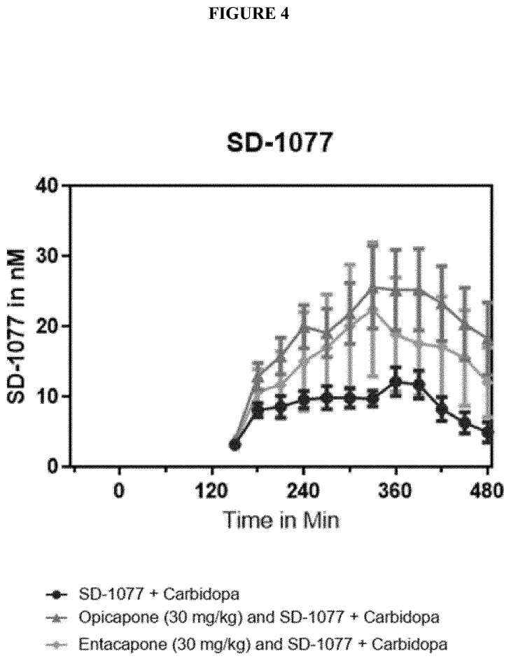

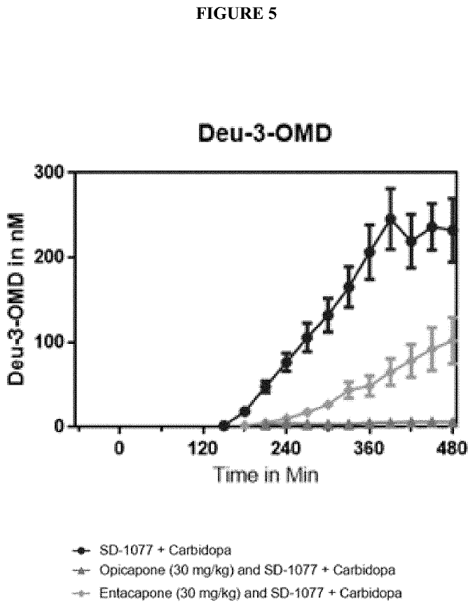

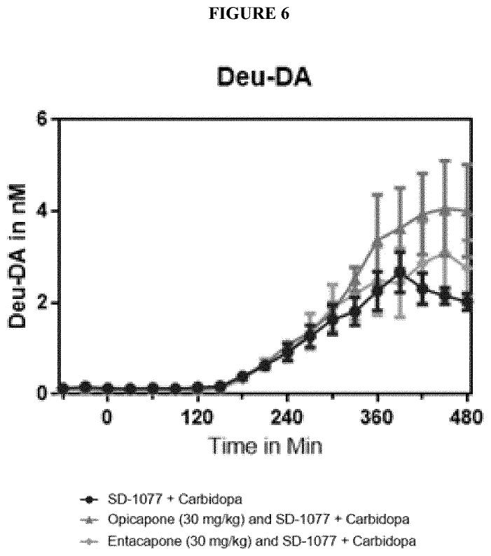

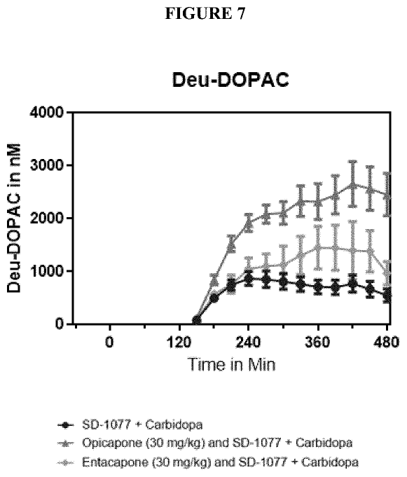

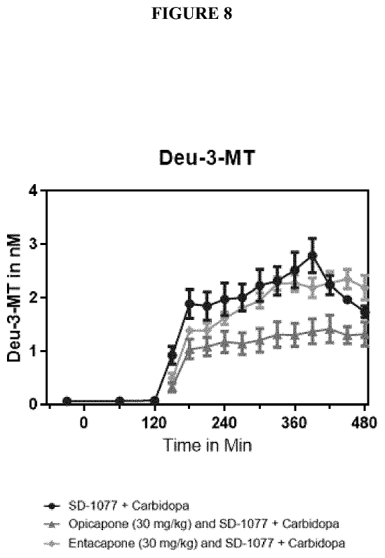

The results of the microdialysis study are depicted in FIGS. 4-8. Opicapone+Composition 1 (SD-1077) demonstrates superiority over Entacapone+Composition 1 (SD-1077). With Opicapone+Composition 1 (SD-1077), higher levels of dopamine and DOPAC and lower levels of 3-OMD and 3-MT were observed.

Opicapone was more effective than entacapone in maintaining the extracellular striatal levels of deuterated L-DOPA following administration of Composition 1 (SD-1077). Opicapone+Composition 1 displayed a higher efficacy in reducing the striatal levels of 3-OMD. Opicapone+Composition 1 was shown to be more potent than Entacapone+Composition 1 in increasing the extracellular levels of deuterated dopamine and its metabolite DOPAC. In line with this, the levels of labeled 3-MT, which derives from dopamine breakdown by COMT, was prominently reduced with Opicapone+Composition 1 while only a mild effect was observed with 30 mg/kg of Entacapone+Composition 1.

### Clinical Trials

One study design for the assessment of combination therapy with deuterated levodopa derivatives (such as a compound of any of structural Formulae I-IV, IIa, IIIa, for example Composition 1) and opicapone consists of a clinical study to investigate the effects of opicapone on deuterated levodopa. Outcome measures are pharmacokinetics, tolerability/safety, and motor performance in advanced PD patients with motor fluctuations.

The study is randomized, multicentre, double-blind, including two parallel arms, and may consist of a screening period, a baseline period (e.g., 4 weeks) and a treatment period (e.g. 3 months) followed by a follow-up period (e.g., 2-weeks). Fifty PD patients treated with standard-release levodopa/carbidopa and with motor fluctuations including dyskinesia are switched to treatment with deuterated levodopa/carbidopa in a 4-week pre-phase of the study. The dose of deuterated levodopa may be reduced by ca. 40% and individual adaptations are required. At the end of the 4-week pre-phase the patients are on a stable deuterated levodopa/carbidopa therapy.

The patients are then randomized to placebo or opicapone 20 mg “add on” applied once daily in the morning with the first dose of deuterated levodopa. Instruction may be given to administer opicapone without food (i.e., in a fasted state) since a marked food effect has been observed, significantly decreasing the rate and extent of opicapone absorption. Additionally, instruction may be given to administer opicapone before levodopa/Composition 1, since a decrease in levodopa Cₘₐₓ, an increase in levodopa systemic exposure, and a more sustained absorption of levodopa have been observed upon sequential dosing when compared to the increase observed with concomitant administration. Instruction may also be given to administer opicapone prior to sleep. See, e.g., US2014/0045900A1. After start of the “add on” treatment, another phase of dose adaptation is required. The patients stay on study treatment for 3 months, with neurological examinations and safety assessments (visits) at 2, 4, 8 and 12 weeks (endpoint).

Subjects.

Subjects may include males and females (with non-childbearing potential) diagnosed with idiopathic PD, defined by the presence of at least two of the cardinal signs of the disease (bradykinesia and at least one of muscular rigidity, rest tremor and postural instability), without any other known cause of parkinsonism and a modified Hoehn and Yahr stage of <5 in the OFF state may be selected for the study. See, e.g., Goetz C G, et al., “Movement Disorder Society Task Force report on the Hoehn and Yahr staging scale: status and recommendations,” Mov Disord 2004; 19: 1020-1028. Patients with PD onset at younger than 30 years or previously treated with entacapone or tolcapone may be excluded. Patients may have had to receive optimum and stable (3-8 daily doses) levodopa therapy, notwithstanding the predictable signs of end-of-dose deterioration (wearing-OFF type) including the presence of at least 1.5 h OFF time during the waking day. Patients whose antiparkinsonian therapy was adjusted within 4 weeks prior to randomization may be excluded.

Assessments.

Throughout the study, vital signs may be recorded, blood sampled for determination of plasma drug concentrations and enzymatic activity and other assessments made, e.g. Clinical and Patient Global Assessment of Change, Unified Parkinson's Disease Rating Scale (UPDRS), and/or modified Abnormal Involuntary Movement Scale (AIMS)]. Subjects complete diaries to record motor fluctuations (periods of ON, OFF, and dyskinesias) throughout the study (to be completed weekly, for example).

Safety Assessments.

Safety and tolerability assessments may include routine laboratory tests (blood chemistry, hematological profile, coagulation and urinalysis), physical examination, electrocardiogram (ECG) and vital signs. Any undesirable sign, symptom or medical condition occurring after starting the study, whether reported spontaneously or when prompted, is typically recorded regardless of suspected relation to the study medication. UPDRS part IV (complications of therapy in the past week) may also be assessed at admission to period 1 and discharge from period 2 and modified AIMS before each test and at its best-ON.

Pharmacokinetics.

Blood samples for PK analyses of levodopa/Composition 1, opicapone, and any relevant metabolites may be taken, and determination of plasma concentrations assessed by, e.g., liquid chromatography with electrochemical or tandem mass detection using a validated method with an appropriate lower limit of quantification

Efficacy: Motor Response Base on Levodopa Tests.

The so-called levodopa test may be modified from that adopted by the Core Assessment Program for Intracerebral Transplantations Committee. The time to ON (interval between time of ON start after test dose and time of test dose intake), time to best-ON (interval between time of best-ON start after test dose and time of test dose intake) and the ON duration (interval between ON onset and the onset of wearing-OFF after the test dose) after each test dose are recorded.

Efficacy: Motor Response Based on Patients' Diaries.

During both periods 1 and 2, subjects keep a daily diary to record ON/OFF periods. For each 30-min period during the day, subjects (with the help of a caregiver, if needed) rate their mobility as OFF (poor mobility or complete immobility), ON with troublesome dyskinesia (limited mobility), ON with non-troublesome dyskinesia (good mobility), ON without dyskinesia (excellent mobility) and asleep.

Efficacy: Unified Parkinson's Disease Rating Scale.

Unified Parkinson's Disease Rating Scale parts I, II (at ON and OFF), III, V and VI may be completed at admission to period 1 and discharge from period 2. UPDRS part III may also be applied before each levodopa test dose and at its best-ON.

Efficacy: Investigators' and Patients' Global Impression of Change.

At discharge from period 2, both investigators and patients may assess the global patient condition in relation to period 1 as very much improved, much improved, minimally improved, no change, minimally worse, much worse or very much worse.

Analyses.

Appropriate analyses may be designed for each of the above efficacy assessments.

Results.

At the endpoint visit after 12 weeks of treatment, “add on” opicapone is expected to increase the dose-adapted bioavailability of deuterated levodopa by 25% compared to placebo as determined by pharmacokinetic sampling. The dose reduction after “add on” of opicapone is expected to be about 25% compared to about 5% after placebo “add on”. In addition, the patients in the opicapone group are expected to have an increase of ON time without dyskinesia by about 40 min compared to placebo. The side effect profile of both arms is not expected to be able to be differentiated.

These results are expected to prove that the addition of opicapone to deuterated levodopa further improves the advantages of its therapeutic potential with regard to reduced drug exposure and increased ON time without induction of additional dyskinesias.

In certain embodiments, this efficacy is expected to surpass what would be seen with the nondeuterated catecholamine derivative, e.g. levodopa, yielding: a greater increase in the extent of exposure to levodopa/Composition 1 (as assessed by AUC); more frequent and longer motor ON periods; reduced duration of OFF; improvements as assessed by the UPDRS; improved patient and clinician global impression of change; reduced AIMS scores; and by patients' diaries.

Each of the in vivo methods above is expected to yield reduced fluctuation (“smoothening” of steady state) of striatal dopamine following administration of a deuterated levodopa derivative such as Composition 1 in combination opicapone (optionally along with an AADCi such as carbidopa or benserazide). The combination of a deuterated levodopa derivative such as Composition 1 and opicapone is expected to yield a greater smoothening effect than that which is seen with a deuterated levodopa derivative alone, both in comparison to non-deuterated levodopa therapy. The smoothening effect will be most apparent in a patient taking three or more doses of levodopa or derivative per day.

From the foregoing description, one skilled in the art can ascertain the essential characteristics of this invention, and without departing from the spirit and scope thereof, can make various changes and modifications of the invention to adapt it to various usages and conditions.

1. A method of (i) treating a dopamine deficiency disorder in a subject in need thereof, (ii) improving motor ON time without dyskinesia in a patient with Parkinson's disease, (iii) reducing dyskinesia in a subject with a dopamine deficiency disorder, or (iv) reducing motor OFF time in a subject with a dopamine deficiency disorder comprising administering to the subject, concurrently or in any order, opicapone and a deuterated levodopa derivative.

2. The method of claim 1, wherein the deuterated levodopa derivative has Formula I: 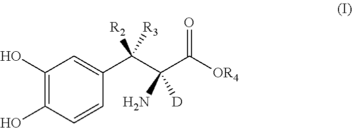
or a stereoisomer, salt, solvate, or prodrug thereof, wherein:
R₂ and R₃ are independently selected from hydrogen and deuterium, and at least one of R₂ and R₃ has a deuterium enrichment in the range from 0.02% to 100% deuterium; and wherein the deuterium enrichment of R₂ and R₃ is different from each other and that the difference between the deuterium enrichment of R₂ and R₃ is at least 5 percentage points; and
R₄ is hydrogen, deuterium, C₁ to C₆-alkyl or C₅ to C₆-cycloalkyl, deuterated C₁ to C₆-alkyl or C₅ to C₆-cycloalkyl, or a group that is easily hydrolytically or enzymatically cleavable under physiological conditions.

3. The method of claim 1, that is for improving motor ON time without dyskinesia in a patient with Parkinson's disease.

4. The method of claim 3, wherein the deuterated levodopa derivative has Formula I: 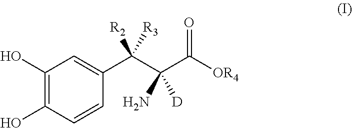
or a stereoisomer, salt, solvate, or prodrug thereof, wherein:
R₂ and R₃ are independently selected from hydrogen and deuterium, and at least one of R₂ and R₃ has a deuterium enrichment in the range from 0.02% to 100% deuterium; and wherein the deuterium enrichment of R₂ and R₃ is different from each other and that the difference between the deuterium enrichment of R₂ and R₃ is at least 5 percentage points; and
R₄ is hydrogen, deuterium, C₁ to C₆-alkyl or C₅ to C₆-cycloalkyl, deuterated C₁ to C₆-alkyl or C₅ to C₆-cycloalkyl, or a group that is easily hydrolytically or enzymatically cleavable under physiological conditions.

5. The method of claim 1, that is for reducing dyskinesia in a subject with a dopamine deficiency disorder.

6. The method of claim 5, wherein the deuterated levodopa derivative has Formula I: 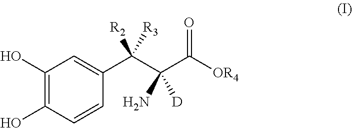
or a stereoisomer, salt, solvate, or prodrug thereof, wherein:
R₂ and R₃ are independently selected from hydrogen and deuterium, and at least one of R₂ and R₃ has a deuterium enrichment in the range from 0.02% to 100% deuterium; and wherein the deuterium enrichment of R₂ and R₃ is different from each other and that the difference between the deuterium enrichment of R₂ and R₃ is at least 5 percentage points; and
R₄ is hydrogen, deuterium, C₁ to C₆-alkyl or C₅ to C₆-cycloalkyl, deuterated C₁ to C₆-alkyl or C₅ to C₆-cycloalkyl, or a group that is easily hydrolytically or enzymatically cleavable under physiological conditions.

7. The method of claim 1, that is for reducing motor OFF time in a subject with a dopamine deficiency disorder.

8. The method of claim 7, wherein the deuterated levodopa derivative has Formula I: 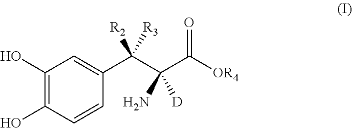
or a stereoisomer, salt, solvate, or prodrug thereof, wherein:
R₂ and R₃ are independently selected from hydrogen and deuterium, and at least one of R₂ and R₃ has a deuterium enrichment in the range from 0.02% to 100% deuterium; and wherein the deuterium enrichment of R₂ and R₃ is different from each other and that the difference between the deuterium enrichment of R₂ and R₃ is at least 5 percentage points; and
R₄ is hydrogen, deuterium, C₁ to C₆-alkyl or C₅ to C₆-cycloalkyl, deuterated C₁ to C₆-alkyl or C₅ to C₆-cycloalkyl, or a group that is easily hydrolytically or enzymatically cleavable under physiological conditions.

9. The method of claim 1, wherein the treatment comprises reducing striatal dopamine level fluctuations in a subject with a dopamine deficiency disorder.

10. The method of claim 9, wherein the deuterated levodopa derivative has Formula I: 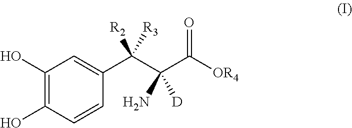
or a stereoisomer, salt, solvate, or prodrug thereof, wherein:
R₂ and R₃ are independently selected from hydrogen and deuterium, and at least one of R₂ and R₃ has a deuterium enrichment in the range from 0.02% to 100% deuterium; and wherein the deuterium enrichment of R₂ and R₃ is different from each other and that the difference between the deuterium enrichment of R₂ and R₃ is at least 5 percentage points; and
R₄ is hydrogen, deuterium, C₁ to C₆-alkyl or C₅ to C₆-cycloalkyl, deuterated C₁ to C₆-alkyl or C₅ to C₆-cycloalkyl, or a group that is easily hydrolytically or enzymatically cleavable under physiological conditions.

11. A pharmaceutical composition comprising a deuterated levodopa derivative and opicapone, together with a pharmaceutically acceptable carrier.

12. The pharmaceutical composition of claim 11, wherein the deuterated levodopa derivative has Formula I: 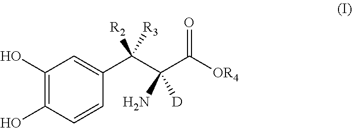
or a stereoisomer, salt, solvate, or prodrug thereof, wherein:
R₂ and R₃ are independently selected from hydrogen and deuterium, and at least one of R₂ and R₃ has a deuterium enrichment in the range from 0.02% to 100% deuterium; and wherein the deuterium enrichment of R₂ and R₃ is different from each other and that the difference between the deuterium enrichment of R₂ and R₃ is at least 5 percentage points; and
R₄ is hydrogen, deuterium, C₁ to C₆-alkyl or C₅ to C₆-cycloalkyl, deuterated C₁ to C₆-alkyl or C₅ to C₆-cycloalkyl, or a group that is easily hydrolytically or enzymatically cleavable under physiological conditions.

13. The pharmaceutical composition of claim 11, wherein the composition comprises an immediate-release portion and a delayed-release portion, and wherein the opicapone is in the immediate release portion, and the deuterated levodopa derivative is in the delayed release portion, such that the deuterated levodopa derivative is absorbed about one hour after absorption of the opicapone.

14. The pharmaceutical composition of claim 11, additionally comprising an AADC inhibitor.

15. The pharmaceutical composition of claim 14, wherein the amount of the deuterated levodopa derivative of Formula I is about 25 to about 200 mg, the amount of opicapone is about 5 to about 50 mg, and the amount of the AADC inhibitor is about 10 to about 50 mg.

16. A method of treating a dopamine deficiency disorder in a subject comprising administering to the subject the pharmaceutical composition of claim 11.

17. A package comprising:
a) the pharmaceutical composition of claim 11; and
b) instructions for use of the pharmaceutical composition to treat a subject afflicted with a dopamine deficiency disorder.

18. The package of claim 17, wherein the deuterated levodopa derivative has Formula I: 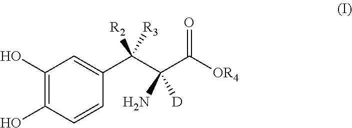
or a stereoisomer, salt, solvate, or prodrug thereof, wherein:
R₂ and R₃ are independently selected from hydrogen and deuterium, and at least one of R₂ and R₃ has a deuterium enrichment in the range from 0.02% to 100% deuterium; and wherein the deuterium enrichment of R₂ and R₃ is different from each other and that the difference between the deuterium enrichment of R₂ and R₃ is at least 5 percentage points; and
R₄ is hydrogen, deuterium, C₁ to C₆-alkyl or C₅ to C₆-cycloalkyl, deuterated C₁ to C₆-alkyl or C₅ to C₆-cycloalkyl, or a group that is easily hydrolytically or enzymatically cleavable under physiological conditions.

19. A method of treating Parkinson's disease in a patient in need thereof, comprising administering to the patient opicapone, carbidopa or benserazide, and Composition 1 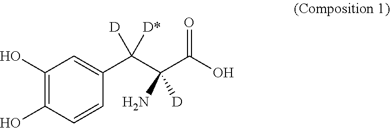
wherein in Composition 1
each position designated D has deuterium enrichment of about 97% or more; and
each position designated D* has deuterium enrichment of about 90%.
